(19) United States
(12) Patent Application Publication
(10) Pub. No.: US 2023/0263736 A1
(43) Pub. Date: Aug. 24, 2023

(54) SOFT GELATIN CAPSULES

(71) Applicant: ITF RESEARCH PHARMA, S.L.U, Madrid (ES)

(72) Inventors: Jaime MOSCOSO DEL PRADO, Madrid (ES); Katia URSO, Madrid (ES); Elena PÉREZ HERNANDO, Madrid (ES)

(73) Assignee: ITF RESEARCH PHARMA, S.L.U, Madrid (ES)

(21) Appl. No.: 18/002,947

(22) PCT Filed: Jun. 24, 2021
(86) PCT No.: PCT/EP2021/067306

§ 371 Date: Dec. 22, 2022

(30) Foreign Application Priority Data
Jun. 24, 2020 (EP) 20382554.2

(51) Int. Cl. A61K 9/48; A61K 31/6615; A61K 31/20; A61K 47/02

(52) U.S. Cl. CPC A61K 9/4825; A61K 31/6615; A61K 31/20; A61K 47/02

(57) ABSTRACT

The present invention relates to soft gelatin capsules, to the use of the soft gelatin capsule as a food supplement, dietary supplement and health supplement, to a process for preparing the soft gelatin capsule, to the soft gelatin capsule for use in medicine and to the soft gelatin capsule for use in the treatment and/or prevention of a disease or pathology.

### FIELD OF THE INVENTION

The present invention relates to soft gelatin capsule comprising choline glycerophosphate and at least an essential unsaturated fatty acid.

### STATE OF THE ART

Choline is a vitamin-like essential nutrient required for neurotransmitters synthesis, biosynthesis and transport of lipids, detoxification mechanisms and membrane metabolism. (Zeisel et al, Nutr. Rev. 2009 November; 67(11): 615-623) For example, the body needs choline to synthesize phosphatidylcholine and sphingomyelin, two major phospholipids vital for cell membranes. Consequently, choline insufficiency can cause muscle damage, liver damage, and nonalcoholic fatty liver disease (NAFLD or hepatosteatosis). The following groups are among those most likely to need coline: pregnant women, people with certain genetic alterations and patients requiring total parenteral nutrition. Preclinical studies have shown that choline is necessary for the development of the brain, neural tube and the entire nervous system of the fetus (Mudd et al, Curr Dev Nutr 2018; 2). Pregnant women in the lowest quartile for dietary choline intake, have four times more risk (compared with women in the highest quartile) of having a baby with a neural tube defect. Moreover, maternal intake of choline has an impact on the neurodevelopment of the fetus, especially improving visual memory, processing speed, attention and better performance of the babies and children. For the mother, adequate intake of choline is also required to support placental function (Mun et al, Nutrients 2019, 11, 1125; Korsmo et al, Nutrients 2019, 11, 1823). In addition, during lactation, choline is produced in breast milk and keeps contributing to the growth and development of the infants (Korsmo et al, Nutrients 2019, 11, 1823). As a consequence, choline demand in pregnant and breastfeeding women is increased and the recommended daily intake is higher. Importantly, epidemiology studies have highlighted a deficit of choline intake among the population of pregnant and breastfeeding women, therefore, justifying the design of a food supplement or a pharmaceutical formulation containing choline. (Davenport et al, J Nutr Biochem. 2015 September; 26(9):903-11; EFSA Report, 16 Aug 2017) Choline is naturally present in some foods and it is also available as a choline only dietary supplement and in some multivitamin/multimineral products. The forms of choline in dietary supplements generally include choline bitartrate, phosphatidylcholine, and lecithin.

Docosahexaenoic acid (DHA) is required for the brain function by modulating neurogenesis, influencing neurotransmission and promoting synaptic activity. Additionally, DHA participates in other physiological processes important for human health, including cell signaling, lipid metabolism and cell membrane function, and is important for eye development and function (Mun et al, Nutrients 2019, 11, 1125). DHA is particularly essential for a healthy pregnancy and for the development of the fetus and child. During pregnancy, adequate DHA supplementation reduces the risk of preterm birth, low birthweight, perinatal death or need of neonatal intensive care (Middleton et al, Cochrane Database of Systematic Reviews 2018, Issue 11. Art. No.: CD003402). Maternal DHA status during fetal development can have lasting impacts on brain and eye health throughout life. In fact, it is recommended to ensure adequate DHA intakes during the first 1000 days of life precisely because DHA is essential for normal vision and brain development (Mun et al, Nutrients 2019, 11, 1125).

Food supplements are becoming increasingly popular as concentrated sources of nutrients. They comprise a wide range of nutrients and other ingredients with a nutritional or physiological effect and are marketed in “dose” form (e.g. pills, tablets, capsules, liquids in measured doses). Food supplements are intended to correct nutritional deficiencies, maintain an adequate intake of certain nutrients, or to support specific physiological functions.

However, the development of food supplements or medicaments comprising choline is challenging due to the difficulty of including the adequate amount of choline in a capsule of acceptable size for patients and, additionally, because of the low stability over time of some sources of choline, particularly in combination with other compounds such as vitamins and minerals. Therefore, there is a clear need for new dietary supplements or medicaments comprising choline in capsule form with adequate size, high stability over time and reduced manufacturing cost.

### SUMMARY OF THE INVENTION

Compared to other ways of administration, the use of soft capsules through oral route have the advantages of being very easy to swallow, free from taste and odour and able to protect the filling from oxidation and degradation. However, the development of formulations comprising choline in soft capsule form is challenging.

On the one hand, the choline contribution of some sources of said compound, like lecithin or phosphatidylcholine, is very small in relation to its total mass. Thus, the amount of these choline sources that should be added to reach an adequate amount of choline would make the capsules enormous and difficult to swallow. This disadvantage is particularly relevant when other active ingredients in high amount, such us a source of unsaturated fatty acids and/or vitamins and minerals, have to be included in the capsules as well. Additionally, undesirable chemical interactions can occur in these complex mixtures that may lead to a reduced stability and, therefore, potency of the nutrients.

On the other hand, other sources of choline, such as choline bitartrate, can be particularly unstable when encapsulated in soft capsules, especially in soft gelatin capsules. Mostly, soft capsules might break and/or suffer changes of shape and color under storage conditions. In addition, choline glycerophosphate, the same as other sources of choline, is a highly hygroscopic compound, which entails technological and stability problems, particularly when it is present in high amounts in a composition. So, costly and complex fabrication processes are needed to prepare soft capsules filled with choline.

Surprisingly, the authors of the present invention have found that the use of choline glycerophosphate (GPC) as source of choline in soft gelatin capsule filling allows to reach an adequate amount of choline using a reasonable amount of GPC and lead to an improved capsule stability under storage in spite of its high hygroscopicity. The physical and organoleptic stability of the capsules are maintained even when choline glycerophosphate is present in a composition with other compounds, particularly unsaturated fatty acids, vitamins and/or minerals, in the filling of the capsule. Additionally, the chemical stability of the active ingredients is also maintained even though the presence of water adsorbed by the GPC may contribute to degradation either directly or by facilitating interactions such as redox reactions between the components.

Thus, the present invention is directed to a soft gelatin capsule comprising a filling comprising choline glycerophosphate, to the use of the soft gelatin capsule as food or dietary supplement, to the soft gelatin capsule for use in medicine and to a process for preparing the soft gelatin capsule.

Therefore, according to a first aspect, the present invention is directed to a soft gelatin capsule comprising:
a) a soft gelatin capsule shell; and
b) a capsule filling composition comprising:
   between 50 and 1000 mg choline glycerophosphate,
   at least an essential unsaturated fatty acid; and
   at least a humidity sensitive vitamin.

Another aspect of the present invention is directed to the non-therapeutic use of the soft gelatin capsule as defined above as a food supplement, dietary supplement, health supplement or nutraceutical; particularly for a subject suffering or who could suffer from choline and/or DHA and/or vitamins insufficiency; particularly in a pregnant woman, a lactating woman, a fetus, infant or children.

According to a further aspect, the present invention is directed to a soft gelatin capsule as defined above, for use in medicine, particularly in preventive medicine and/or preventive healthcare.

A further aspect of the invention is directed to a soft gelatin capsule as defined above, for use in the treatment and/or prevention of a disease or pathology related to choline and/or DHA and/or vitamins insufficiency, particularly wherein the subject suffering or who could suffer from said disease or pathology is a pregnant woman, a lactating woman, a fetus, an infant or a child.

In an embodiment, the soft gelatin capsule is used as a food supplement, dietary supplement, health supplement or nutraceutical.

A further aspect of the present invention is directed to a process for preparing the soft gelatin capsule of the invention, said process comprising:
a) preparing a capsule filling composition comprising
   between 50 and 1000 mg choline glycerophosphate,
   at least an essential unsaturated fatty acid; and
   at least a humidity sensitive vitamin;
b) forming a soft gelatin capsule shell;
c) filling the soft gelatin capsule shell with the capsule filling composition of step a) to obtain a soft gelatin capsule, and
d) drying the soft gelatin capsule obtained in step c).

A further optional aspect of the present invention is directed to a soft gelatin capsule obtainable by the process of the invention in any of its particular embodiments.

### DESCRIPTION OF THE FIGURES

FIG. 1 shows a blister comprising the soft gelatin capsules after 24 months stored at 25° C. and 60% of RH.

FIG. 2 shows an image of a broken soft gelatin capsule blister.

FIG. 3 shows an image of a broken shell of a soft gelatin capsule comprising choline bitartrate.

FIG. 4 shows an image of gelatin capsules wherein the capsule shell has changed of color.

### DETAILED DESCRIPTION OF THE INVENTION

With regard to the terms used in the present description, unless otherwise defined, scientific and technical terms used in connection with the present invention shall have the meanings that are commonly understood by those of ordinary skill in the art. The meaning and scope of the terms should be clear, however, in the event of any latent ambiguity, definitions provided herein take precedent over any dictionary or extrinsic definition.

As used herein, the terms “about” or “around” means a slight variation of the value specified, preferably within 10 percent of the value specified. Nevertheless, the term “about” or the term “around” can mean a higher tolerance of variation depending on for instance the experimental technique used. Said variations of a specified value are understood by the skilled person and are within the context of the present invention. Further, to provide a more concise description, some of the quantitative expressions given herein are not qualified with the term “about”. It is understood that, whether the term “about” is used explicitly or not, every quantity given herein is meant to refer to the actual given value and it is also meant to refer to the approximation to such given value that would reasonably be inferred based on the ordinary skill in the art, including equivalents and approximation due to the experimental and/or measurement conditions for such given value.

In the context of the present invention, the term “wt %” means weight percent; it is generally expressed regarding the total weight of the capsule unless it is otherwise indicated in the text.

### Soft Gelatin Capsules

According to a first aspect, the present invention is directed to a soft gelatin capsule comprising:
a) a soft gelatin capsule shell; and
b) a capsule filling composition comprising:
   between 50 and 1000 mg choline glycerophosphate,
   at least an essential unsaturated fatty acid; and
   at least a humidity sensitive vitamin.

In a particular embodiment, the total weight of the soft gelatin capsule is in between 300 and 2000 mg; preferably between 500 mg and 1900 mg; more preferably between 800 and 1800 mg.

In a particular embodiment, the soft gelatin capsule comprises choline glycerophosphate in an amount between 50 and 1000 mg; particularly between 50 and 900 mg; more particularly between 60 and 800 mg; preferably in between 70 and 700 mg; more preferably in between 100 and 600 mg.

In a particular embodiment, the soft gelatin capsule comprises at least an essential unsaturated fatty acid in an amount between 50 and 900 mg; preferably in between 70 and 700 mg; more preferably 100 and 500 mg; preferably at least DHA.

### Capsule Shell

The soft gelatin capsule of the invention comprises a soft gelatin capsule shell. The term “soft capsule” is well known in the art and refers to a capsule having a soft capsule shell, as opposed to hard capsules that are made up of a rigid shell. In the present invention the soft capsule is a soft gelatin capsule wherein the shell comprises gelatin in its composition. A soft capsule shell may generally be made by forming a mixture comprising gelatin, water and a plasticizer in different ratios and then drying them. Soft gelatin capsules may be a single case produced from gelatin, rather than two halves attached together. In addition, they may be used for oil based solutions wherein the active ingredients are dissolved or dispersed. The soft gelatin capsule may be manufactured and filled using the same machine as part of a single process. Hard gelatin capsules, unlike the soft capsules form, are generally made of two parts, the body and the cap. This form of capsule usually holds dry ingredients in powder form, in pellets or in form of premade minitablets. The body of the hard gelatin capsules might be first filled with the mix of active ingredients and any excipients used, and then closed with the cap using either a manual or automatic press machine.

The soft gelatin capsule shell may be any of those approved for pharmaceutical or nutritional use, preferably made of gelatin, at least a plasticizer, and optional ingredients such as opacifying agents, flavors and colorants.

The gelatin suitable for making the soft gelatin capsule shell of the soft gelatin capsule of the invention is any of those approved by local authorities for pharmaceutical or nutritional use. These gelatins are mainly of two different types either alkaline (Type B) or acid (Type A) with medium gel strength (medium Bloom, such as 150-200 Bloom). Both types may be used in combination or separately. Non limiting examples of type A gelatins are pig skin gelatin, acid hide gelatin and fish gelatin. Non limiting examples of gelatin B are limed bone gelatins, limed hide gelatin and bovine skin gelatin.

In a particular embodiment, the soft gelatin capsule shell of the soft gelatin capsule of the invention comprises gelatin B, preferably bovine gelatin and/or gelatin A, preferably pig skin gelatin or fish gelatin.

In an embodiment, the soft gelatin capsule of the invention comprises gelatin in an amount between 50 and 500 mg; preferably in between 100 mg and 350 mg.

In an embodiment, the soft gelatin capsule of the invention comprises gelatin in an amount of between 5 and 30 wt % of the total weight of the soft gelatin capsule; more preferably between 10 and 25 wt %; even more preferably around 15% wt or around 20 wt %.

In an embodiment the soft capsule shell further comprises a plasticizer selected from the group consisting of glycerine, sorbitol, propylene glycol, polyethylene glycol, dibutyl sebacate, diethyl phthalate, dimethyl phthalate, triacetin, tributyl citrate, triethyl citrate, and mixtures thereof; preferably a plasticizer selected from the group consisting of glycerine, sorbitol and propylene glycol; more preferably glycerine and/or sorbitol.

In an embodiment, the soft gelatin capsule of the invention comprises a plasticizer in an amount between 5 and 300 mg; preferably in between 10 and 150 mg.

In an embodiment, the soft gelatin capsule of the invention comprises a plasticizer in an amount between 1 and 20 wt % of the total weight of the soft gelatin capsule; more preferably in between 2 and 10 wt %.

In an embodiment the soft capsule shell further comprises an opacifying agent and/or a coloring agent; preferably a coloring agent.

In the context of the present invention the term “opacifying agents” relates to agents that may be added to the soft gelatin capsule shell in order to make the shell opaque and thus protect the soft capsule filling composition from light. Suitable opacifying agents are known in the art and include titanium dioxide, talc, and the like.

In a particular embodiment, the soft capsule shell does not comprise an opacifying agent, preferably it does not comprise titanium dioxide.

In the context of the present invention the term “coloring agents” relates to agents that may be added to the soft capsule shell to give the shell the desired color. Suitable coloring agents are known in the art and include sunset yellow FCF (E-110), carmine red (E-120), indigo carmine (E-132), erythrosine (E-127), quinoline yellow (E-104), iron oxide (E-172) and the like; preferably sunset yellow FCF (E-110), indigo carmine (E-132) and iron oxide (E172); more preferably iron oxide (E172).

In an embodiment, the soft gelatin capsule of the invention comprises at least one coloring agent in between 0.01 and 10 mg; preferably 0.05 and 7 mg.

In an embodiment, the soft gelatin capsule of the invention comprises at least one coloring agent in an amount between 0.10 and 0.50 wt % of the total weight of the soft gelatin capsule; more preferably in between 0.15 and 0.35 wt %; even more preferably of about 0.25 wt %.

In another embodiment, the soft gelatin capsule shell comprises:
50 to 500 mg of gelatin,
5 to 300 mg of a plasticizer, and
optionally, 0.01 to 10 mg of a coloring agent.

In another embodiment, the soft gelatin capsule shell comprises:
100 to 350 mg of gelatin,
10 and 150 mg of a plasticizer selected from glycerine and/or sorbitol,
0.05 to 5 mg of a coloring agent, preferably iron oxide.

In another embodiment, the soft gelatin capsule shell comprises:
5 to 30 wt % of gelatin,
1 to 20 wt % of at least one plasticizer, and
optionally, a coloring agent;
   the amounts by weight being expressed with respect to the total weight of the soft gelatin capsule.

In another embodiment, the soft gelatin capsule shell comprises:
10 to 25 wt % of gelatin,
2 to 10 wt % of glycerin and/or 1 to 10 wt % of sorbitol, particularly between 1 to 5 wt % of sorbitol;
0.005 to 1 wt % of a coloring agent; preferably iron oxide;
   the amounts by weight being expressed with respect to the total weight of the soft gelatin capsule.

In another embodiment, the soft gelatin capsule shell weights between 20 and 30 wt % of the soft gelatin capsule; preferably about 30 wt %; more preferably between 100 and 500 mg; even more preferably between 150 and 450 mg.

### Capsule Filling Composition

The soft gelatin capsule of the invention comprises a capsule filling composition. The capsule filing composition of the soft gelatin capsule of the invention comprises
between 50 and 1000 mg choline glycerophosphate,
at least an essential unsaturated fatty acid; and
at least a humidity sensitive vitamin.
   Choline glycerophosphate

In the context of the present invention “choline glycerophosphate” (GPC) may be named as choline alfoscerate, as glycerylphosphorylcholine or as bis(2-hydroxy-N,N,N-trimethylethanaminium) 2,3-dihydroxypropyl phosphate (CAS number 28319-77-9).

Any suitable choline glycerophosphate may be used in the soft gelatin capsule of the present invention of those approved for pharmaceutical or nutritional use. In a more particular embodiment, 100 mg of the choline glycerophosphate in the soft gelatin capsule of the present invention provide around 40.5 mg of choline. From this relation the amount of the essential nutrient choline can be calculated from the amount of choline glycerophosphate included in the composition.

In a particular embodiment, the soft gelatin capsule filling composition comprises choline glycerophosphate in an amount between 50 and 1000 mg; particularly between 50 and 900 mg; more particularly between 60 and 800 mg; preferably in between 70 and 700 mg; more preferably in between 100 and 600 mg.

In a more particular embodiment, the choline glycerophosphate in the soft gelatin capsule or filling composition is between 200 and 600 mg; more preferably around 200, 225, 230, 235, 240, 245, 250, 275, 300, 325, 350, 400, 450, 500, 550 or 600 mg.

In a more particular embodiment, the choline glycerophosphate in the soft gelatin capsule or filling composition is between 100 and 300; more preferably around 100, 110, 120, 130, 140, 160, 180, 200, 250 or 300 mg; even more preferably around 100 or 200 mg.

In a particular embodiment, the choline glycerophosphate in the soft gelatin capsule is in at least a 2.5 wt % of the total weight of the soft gelatin capsule; preferably at least a 3 wt %; more preferably at least a 4 wt %; even more preferably at least a 5 wt %, a 6 wt %, a 7 wt % or a 8 wt % of the total weight of the soft gelatin capsule; preferably in at least a 8.5 wt %; more preferably in at least a 9 wt %; even much more preferably in at least a 10 wt %.

In another particular embodiment, the choline glycerophosphate in the soft gelatin capsule is between 2.5 and 85 wt % of the total weight of the soft gelatin capsule; particularly in between 3 and 85 wt %; preferably in between a 3 and a 82 wt %; more preferably in between 3.5 and 80 wt %; even more preferably between 8 and 70 wt %; preferably between 8 and 65% wt %; more preferably between 9 and 65 wt %; more particularly between 9 and 60 wt %; even more preferably between 10 and 60 wt %; more particularly between 10 and 50 wt %.

In a particular embodiment, the choline glycerophosphate in the soft gelatin capsule is between 10 and 50 wt %, preferably between 15 and 35 wt % of the total weight of the soft gelatin capsule.

In a particular embodiment, the choline glycerophosphate in the soft gelatin capsule is between 8 and 35 wt %, preferably between 10 and 20 wt % of the total weight of the soft gelatin capsule.

In another particular embodiment, the soft gelatin capsule of the invention comprises 25 less than 15 wt % of the total weight of the soft gelatin capsule of other sources of choline different from choline glycerophosphate; preferably less than 10 wt %. In the context of the present invention other sources of choline comprises choline bitartrate, cytidine diphosphate choline (CDP-choline; citicoline), phosphatidylcholine, and lecithin; in particular choline bitartrate.

### Fatty Acids

The capsule filling composition further comprises at least an essential unsaturated fatty acid.

In the context of the present invention, the term “essential unsaturated fatty acid” refers to a straight chain hydrocarbon possessing a carboxyl group at one end having one or more double bonds (C═C), preferably one, two, three, four, five or six double bonds, wherein the first double bond exists at the third carbon-carbon bond from the terminal CH₃ end of the carbon chain (omega-3 fatty acid), at the sixth carbon-carbon bond from the terminal CH₃ end of the carbon chain (omega-6 fatty acid), or at the ninth carbon-carbon bond from the terminal CH₃ end of the carbon chain (omega-9 fatty acid). Non-limiting examples of essential unsaturated fatty acids are omega-3 fatty acids, such as α-linolenic acid (ALA), eicosapentaenoic acid (EPA), and docosahexaenoic acid (DHA), hexadecatrienoic acid (HTA), stearidonic acid (SDA), eicosatrienoic acid (ETE), eicosatetranoic acid (ETA), heneicosapentaenoic acid (HPA), docosapentaenic acid (DPA), tetracosapentaenoic acid and tetracosahexaenoic acid and mixtures thereof; omega-6 fatty acids, such as linoleic acid (LA), γ-linolenic acid (GLA), calendic acid, eicosadienoic acid, dihomo-γ-linolenic acid (DGLA), arachidonic acid (AA), docosadienoic acid, adrenic acid, docosapentaenoic acid, tetracosatetraenoic acid, tetracosapentaenoic acid, and mixtures thereof; omega-9 fatty acids, such as oleic acid, elaidic acid, gondoic acid, mead acid, erucic acid and nervonic acid, and mixtures thereof.

In a particular embodiment, the at least an essential unsaturated fatty acid of the invention is at least an omega-3 fatty acid selected from α-linolenic acid (ALA), eicosapentaenoic acid (EPA), docosahexaenoic acid (DHA), hexadecatrienoic acid (HTA), stearidonic acid (SDA), eicosatrienoic acid (ETE), eicosatetranoic acid (ETA), heneicosapentaenoic acid (HPA), docosapentaenic acid and mixtures thereof; wherein said omega-3 fatty acid is a free acid; preferably α-linolenic acid (ALA), eicosapentaenoic acid (EPA), docosahexaenoic acid (DHA), hexadecatrienoic acid (HTA); more preferably it is eicosapentaenoic acid (EPA) and docosahexaenoic acid (DHA).

In a particular embodiment, the essential unsaturated fatty acid is a mixture of essential unsaturated fatty acids. In a particular embodiment, the at least an essential unsaturated fatty acid of the invention is a mixture comprising docosahexaenoic acid (DHA); preferably a mixture comprising eicosapentaenoic acid (EPA) and docosahexaenoic acid (DHA); preferably wherein the mixture EPA and DHA has a weight ratio of EPA:DHA of between 1:50 and 1:1.2; preferably of between 1:20 and 1:2; more preferably about 1:5.

In a particular embodiment, the at least an essential unsaturated fatty acid is a free essential unsaturated fatty acid.

In an embodiment, the capsule filling composition comprises less than 1% of the total weight of the soft gelatin capsule of a mixture of free essential unsaturated fatty acids; preferably less than 0.5 wt %; more preferably less than 0.3 wt %. The amount of free fatty acids in the capsule filling composition may be measured by its acid value (AV) defined as the weight of KOH in mg needed to neutralize the organic acids present in 1 g of fat.

In an embodiment, the at least essential unsaturated fatty acid of the capsule filling composition is an esterified essential unsaturated fatty acid; preferably an esterified essential unsaturated fatty acid part of monoacylgycerols, diacylgycerols, triacylgycerols, ethyl esters and combinations thereof. In an embodiment, the at least essential unsaturated fatty acid of the capsule filling composition is as a mixture of esterified essential unsaturated fatty acid and essential unsaturated fatty acids.

### Fatty Esters

In a particular embodiment the capsule filling composition further comprises at least an essential unsaturated fatty ester; preferably wherein said esters comprise alkylesters, monoglycerides, diglycerides, triglycerides and/or mixtures thereof.

In a particular embodiment, at least an essential unsaturated fatty ester is an ethyl ester.

Non-limiting examples of essential unsaturated fatty esters are those derived of the reaction of one, two or three essential unsaturated fatty acid as defined above with glycerol or with an alkyl alcohol, particularly ethanol; preferably wherein essential unsaturated fatty acid are omega-3 fatty acids; more preferably α-linolenic acid (ALA), eicosapentaenoic acid (EPA), docosahexaenoic acid (DHA), hexadecatrienoic acid (HTA); more preferably are eicosapentaenoic acid (EPA) and docosahexaenoic acid (DHA); even more preferably docosahexaenoic acid (DHA); even much more preferably in a weight ratio of EPA:DHA of between 1:50 and 1:1.2; preferably of between 1:20 and 1:2; more preferably about 1:5, as calculated from the free fatty acids present in the esters.

In a particular embodiment, the capsule filling composition comprises a mixture of essential unsaturated fatty acids and essential unsaturated fatty esters; preferably wherein said esters comprise ethyl esters, monoglycerides, diglycerides, triglycerides and/or a combination thereof.

In an embodiment, the the soft gelatin capsule comprises equal to or less than 1 wt % of the total weight of the soft gelatin capsule of free essential unsaturated fatty acids.

In a particular embodiment, the capsule filling composition comprises a mixture of essential unsaturated fatty esters; wherein said mixture of essential unsaturated fatty esters comprises esters derived from eicosapentaenoic acid (EPA) and docosahexaenoic acid (DHA); preferably wherein the mixture EPA and DHA has a weight ratio of EPA:DHA of between 1:50 and 1:1.2; preferably of between 1:20 and 1:2; more preferably about 1:5 (as calculated from the free fatty acids present in the esters); preferably wherein said esters comprise ethyl esters, monoglycerides, diglycerides, triglycerides and/or a combination thereof.

No-limiting suitable essential unsaturated fatty acids, essential unsaturated fatty esters or mixtures thereof for the embodiments of the present invention can be derived from algae, fish, animals, plants, or combinations thereof; preferably from natural oils derived from these organisms.

In some embodiments, suitable essential unsaturated fatty acids, essential unsaturated fatty esters or mixtures thereof can be provided in fish oils, (e.g., menhaden oil, tuna oil, salmon oil, bonito oil, and cod oil), microalgae omega-3 oils or combinations thereof. In particular embodiments, suitable omega-3 fatty acids may be derived from commercially available omega-3 fatty acid oils such as Microalgae DHA oil (from Martek, Columbia, Md.), OmegaPure (from Omega Protein, Houston, Tex.), Marinol C-38 (from Lipid Nutrition, Channahon, Ill.), Bonito oil and MEG-3 (from Ocean Nutrition, Dartmouth, NS), Evogel (from Symrise, Holzminden, Germany}, Marine Oil, from tuna or salmon (from Arista Wilton, Conn.), OmegaSource 2000, Marine Oil, from menhaden and Marine Oil, from cod (from OmegaSource, RTP, NC).

In a more particular embodiment, the capsule filling composition comprises between 50 mg and 2000 mg of oil; preferably between 100 and 1500 mg; more preferably between 150 and 1000 mg; particularly between 170 and 750 mg; even more preferably between 180 and 750 mg; preferably of fish oil (e.g., menhaden oil, tuna oil, salmon oil, bonito oil, cod oil and mixtures thereof), more preferably of fish oil with a DHA content between 35 and 85 wt %, preferably around 50 wt %.

In a particular embodiment the capsule filling composition comprises fish oil, (e.g., menhaden oil, tuna oil, salmon oil, bonito oil, cod oil and mixtures thereof); preferably a fish oil in between 5 and 75 wt % of the total weight of the soft gelatin capsule; more preferably between 10 and 65 wt %; even more preferably between 15 and 55 wt %; even much more preferably between 17 and 50 wt %.

In a more particular embodiment, the capsule filling composition comprises fish oil, (e.g., menhaden oil, tuna oil, salmon oil, bonito oil, cod oil and mixtures thereof); preferably a fish oil in between 10 and 50 wt % of the total weight of the soft gelatin capsule; more preferably between 15 and 30 wt % or between 20 and 40 wt %.

In a particular embodiment, the capsule filing composition of the soft gelatin capsule of the invention comprises
between 70 and 700 mg of choline glycerophosphate; and
between 100 and 1500 mg of fish oil comprising eicosapentaenoic acid (EPA) and docosahexaenoic acid (DHA); wherein the weight ratio of EPA:DHA is between 1:50 and 1:1.2; and
at least a humidity sensitive vitamin.

In a particular embodiment, the capsule filling composition comprises between 50 mg and 750 mg of fish oil; more preferably between 50 mg and 600 mg of fish oil, even more preferably between 100 and 600 mg of fish oil, comprising docosahexaenoic acid (DHA); preferably comprising eicosapentaenoic acid (EPA) and docosahexaenoic acid (DHA); more preferably wherein the weight ratio of EPA:DHA is between 1:50 and 1:1.2; preferably between 1:3 and 1:8; optionally, wherein EPA or DHA are in the fish oil as essential unsaturated fatty acids and/or as essential unsaturated fatty esters derived from the fatty acids; preferably wherein said esters comprise ethyl esters, monoglycerides, diglycerides, triglycerides and/or a combination thereof.

In an embodiment, the fish oil comprises less than 1% of the total weight of the soft gelatin capsule of a mixture of free essential unsaturated fatty acids; preferably less than 0.5 wt %; more preferably less than 0.3 wt %. The amount of free fatty acids in the oil may be measured by its acid value (AV) defined as the weight of KOH in mg needed to neutralize the organic acids present in 1 g of fat. In an embodiment, the fish oil comprises a maximum acid value of 1 mg of KOH per gram of fish oil. In an embodiment, 1 g of the fish oil comprises around 90 mg of EPA and 450 mg of DHA. In a particular embodiment, the fish oil is an Omega 3-H™ Fish oil.

In a particular embodiment, the soft capsule filling composition comprises at least DHA in an amount between 50 and 700 mg, preferably in between 70 and 500 mg; particularly between 100 and 500 mg; more preferably DHA in an amount between 50 and 700 mg and EPA in an amount between 10 and 140 mg; even more preferably DHA in an amount between 100 and 500 mg and EPA in an amount between 20 and 100 mg.

In a particular embodiment, the capsule filing composition of the soft gelatin capsule of the invention comprises:
between 70 and 700 mg of choline glycerophosphate;
between 50 and 700 mg of DHA; and
at least a humidity sensitive vitamin.

In a preferred embodiment, the capsule filing composition of the soft gelatin capsule of the invention comprises:
between 100 and 600 mg of choline glycerophosphate; and
between 70 and 500 mg of DHA; wherein the weight ratio of EPA:DHA is between 1:50 and 1:1.2; and
at least a humidity sensitive vitamin.

In a preferred embodiment, the capsule filing composition of the soft gelatin capsule of the invention comprises:
between 70 and 700 mg of choline glycerophosphate; and
between 100 and 1500 mg of fish oil comprising eicosapentaenoic acid (EPA) and docosahexaenoic acid (DHA);
wherein the weight ratio of EPA:DHA is between 1:50 and 1:1.2; and
at least a humidity sensitive vitamin.

### Elements

In a particular embodiment, the capsule filling composition further comprises at least an element selected from nickel, copper, selenium, iron, molybdenum, chromium, iodine, magnesium, manganese, phosphorus, potassium, zinc and/or a combination thereof.

In a particular embodiment, the capsule filling composition further comprises at least a compound comprising an element selected from nickel, copper, selenium, iron, molybdenum, chromium, iodine, magnesium, manganese, phosphorus, potassium, zinc and/or a combination thereof; preferably selected from copper, iron or a combination thereof

In a more particular embodiment the capsule filling composition further comprises at least a metal element selected from nickel, copper, iron, zinc and/or a combination thereof; more preferably from copper, iron, zinc and/or a combination thereof; even more preferably from copper, iron and/or a combination thereof.

In a more particular embodiment the capsule filling composition comprises copper, preferably in an amount between 0.020 and 0.600 wt % of the total weight of the soft gelatin capsule; even more preferably between 0.03 and 0.50 wt %. In a preferred embodiment, the copper is in an amount between 0.05 and 5 mg; preferably between 0.1 and 3 mg; even more preferably between 0.2 and 2 mg.

In a more particular embodiment the capsule filling composition further comprises a compound comprising copper. Non-limiting examples of suitable compounds comprising copper are cupric oxide, cupric sulfate, copper amino acid chelates, and copper gluconate; preferably cupric sulfate; more preferably anhydrous cupric sulfate or cupric sulphate 1-hydrate; even more preferably anhydrous cupric sulfate comprising a 40% of elemental copper of the total weight of anhydrous cupric sulfate or cupric sulphate 1-hydrate comprising about 35.78% of elemental copper. In a particular embodiment the capsule filing composition comprises cupric sulfate; preferably anhydrous cupric sulfate or cupric sulfate 1-hydrate in an amount between 0.5 and 5 mg, preferably between 1 and 3 mg.

In a more particular embodiment the capsule filling composition further comprises iron. In a particular embodiment, the capsule filling composition comprises iron, preferably in an amount between 0.1 and 10 wt % of the total weight of the soft gelatin capsule; more preferably in between 0.3 and 7 wt %; even more preferably between 0.5 and 5 wt %. In a preferred embodiment, the iron is in an amount of between 1 and 100 mg; preferably between 2 and 80 mg; even more preferably between 5 and 35 mg.

In a more particular embodiment the capsule filling composition comprises a compound comprising iron. Non-limiting examples of suitable compounds comprising iron are ferric ammonium citrate, ferric phosphate, ferric pyrophosphate, ferritin, ferrocholinate, ferrous ascorbate, ferrous aspartate, ferrous chloride, ferrous sulfate, ferrous tartrate, ferrous fumarate, ferrous gluconate, ferrous gluceptate, ferrous bisglycinate chelate, ferrous glycine sulfate, ferrous lactate, ferrous oxalate and ferrous succinate; preferably ferrous fumarates, ferrous sulfates, ferrous bisglycinate and ferrous gluconates.

In other preferred embodiment, the capsule filling composition comprises ferrous fumarate; even more preferably ferrous fumarate comprising a 32% of elemental iron of the total weight of ferrous fumarate in an amount between 0.1 and 20 wt % of the total weight of the soft gelatin capsule; more preferably in between 1 and 10 wt %. In other preferred embodiment, the capsule filling composition comprises ferrous fumarate; even more preferably ferrous fumarate comprising a 32% of elemental iron of the total weight of ferrous fumarate in an amount between 15 and 150 mg; particularly between 10 and 150 mg; preferably of between 20 and 100 mg; more preferably of between 15 and 100 mg.

In other preferred embodiment, the capsule filling composition comprises ferrous bisglycinate chelate (such as Ferrochel®) in an amount of between 1 and 10 wt % of the total weight of the soft gelatin capsule; more preferably in between 2 and 8 wt %; even more preferably between 3 and 7 wt %. In other preferred embodiment, the capsule filling composition comprises ferrous bisglycinate chelate (such as Ferrochel®) in an amount of between 1 and 150 mg; more preferably of between 10 and 150 mg; particularly 10 and 100 mg; more particularly between 20 and 100 mg.

In a more particular embodiment, the capsule filling composition further comprises a compound comprising iron encapsulated; preferably iron pyrophosphate microencapsulated (for example Lipofer®) in an amount between 25 and 35 wt % of the total weight of the soft gelatin capsule. In other preferred embodiment, the capsule filling composition comprises iron pyrophosphate microencapsulated (for example Lipofer®) in an amount of between 1 and 1000 mg; preferably in an amount of between 100 and 500 mg.

In a more particular embodiment, the capsule filling composition further comprises zinc, preferably in an amount between 0.01 and 10 wt % of the total weight of the soft gelatin capsule; more preferably in between 0.1 and 5 wt %; even more preferably between 0.2 and 2 wt %. In a preferred embodiment, the zinc is in an amount of between 1 and 30 mg; preferably of between 2 and 20 mg; more preferably of between 3 and 15 mg.

In a more particular embodiment, the capsule filling composition comprises a compound comprising zinc. Non-limiting examples of suitable compounds comprising zinc are zinc oxide, zinc gluconate, zinc sulfate, and zinc acetate; preferably zinc oxide; more preferably zinc oxide comprising a 78 wt % of elemental zinc of the total weight of zinc oxide. In a particular embodiment the capsule filing composition comprises zinc oxide (78 wt % of elemental zinc of the total weight of zinc oxide) in an amount between 1 and 30 mg; preferably 2 and 20 mg.

In a particular embodiment, the capsule filling composition further comprises a compound comprising iron, preferably a compound selected from the group consisting of ferrous bisglycinate chelate, ferrous fumarate, iron pyrophosphate microencapsulated or mixtures thereof; more preferably between 15 and 100 mg of ferrous fumarate and/or between 20 and 100 mg of ferrous bisglycinate chelate

In a preferred embodiment, the capsule filling composition further comprises: zinc and/or iron and optionally copper.

In a particular embodiment, the capsule filling composition further comprises:
a compound comprising zinc;
a compound comprising iron; and
optionally, a compound comprising copper.

In a particular embodiment, the capsule filling composition further comprises:
zinc oxide;
a compound selected from the group consisting of ferrous bisglycinate chelate, iron pyrophosphate microencapsulated or ferrous fumarate; and
optionally, copper sulfate.

In an even more particular embodiment, the capsule filling composition further comprises:
between 1 and 30 mg of zinc oxide;
between 15 and 150 mg of ferrous fumarate; and
optionally, between 0.5 and 5 mg of anhydrous copper sulfate.

In an even much more particular embodiment, the capsule filling composition further comprises:
between 2 and 20 mg of zinc oxide;
between 20 and 100 mg of ferrous fumarate; and
optionally, between 1 and 3 mg of anhydrous copper sulfate.

In another particular embodiment, the capsule filling composition further comprises:
between 1 and 30 mg of zinc oxide;
between 10 and 150 mg of ferrous fumarate and/or between 1 and 150 mg of ferrous bisglycinate chelate; and
optionally, between 0.5 and 5 mg of anhydrous copper sulfate.

In another particular embodiment, the capsule filling composition further comprises:
between 2 and 20 mg of zinc oxide;
between 15 and 100 mg of ferrous fumarate and/or between 20 and 100 mg of ferrous bisglycinate chelate; and
optionally, between 1 and 3 mg of anhydrous copper sulfate.

### Other Elements

In a more particular embodiment the capsule filling composition further comprises selenium, preferably in an amount of between 0.0001 and 0.1 wt % of the total weight of the soft gelatin capsule; more preferably in between 0.0003 and 0.1%; even more preferably in between 0.0005 and 0.1 wt %. In a preferred embodiment, the selenium is in an amount of between 1 and 500 μg (micrograms); preferably between 5 and 100 μg; even more preferably between 10 and 70 μg.

In a more particular embodiment the capsule filling composition comprises a compound comprising selenium. Non-limiting examples of suitable compounds comprising selenium are sodium selenite (Se⁺⁴), sodium selenate (Se⁺⁶), sodium selenide (Se⁻²), and Se⁰, SeMet, SeCys and MeSeCys; preferably sodium selenite; more preferably sodium selenite comprising a 45% of elemental selenium of the total weight of sodium selenite. In a particular embodiment the capsule filing composition comprises sodium selenite (45% of elemental selenium of the total weight of sodium selenite) in an amount between 10 and 200 μg, preferably between 20 and 150 μg.

In a more particular embodiment, the capsule filling composition further comprises iodine, preferably in an amount of between 0.001 and 0.1 wt % of the total weight of the soft gelatin capsule; more preferably in between 0.005 and 0.05 wt %. In a preferred embodiment, the iodine is in an amount of between 10 and 1000 μg; preferably between 30 and 500 μg; more preferably of between 50 and 300 μg.

In a more particular embodiment, the capsule filling composition further comprises at least a compound comprising iodine. Non-limiting examples of suitable compounds comprising iodine are iodide or iodate of potassium, calcium or sodium; preferably potassium iodate or potassium iodide; more preferably potassium iodate comprising around 60 wt % of elemental iodine of the total weight of potassium iodate or potassium iodide comprising around 75 wt % of elemental iodine of the total weight of potassium iodide. In a particular embodiment the capsule filing composition comprises potassium iodate (59.9% I) in an amount between 50 and 500 μg, preferably between 100 and 400 μg, or potassium iodide (76.4% I) in an amount between 100 and 300 μg.

In a more particular embodiment, the capsule filling composition further comprises calcium, preferably in an amount between 2 and 40 wt % of the total weight of the soft gelatin capsule; more preferably in between 5 and 20 wt %; even more preferably about 10 wt %. In a preferred embodiment, the calcium is in an amount of between 50 and 500 mg; preferably between 70 and 250 mg; more preferably around 80, 100, 120, 140, 160, 180, 200 or 220 mg.

In a more particular embodiment, the capsule filling composition further comprises at least a compound comprising calcium. Non-limiting examples of suitable compounds comprising calcium are calcium carbonate, calcium chloride, calcium chloride hexahydrate, calcium citrate, calcium formate, calcium glycinate, bisglycinate, calcium glucoheptonate, calcium gluconate, calcium gluconolactate, calcium glutamate, calcium glycerophosphate, calcium hydrogenophosphate, calcium lactate, calcium lactobionate, calcium lactophosphate, calcium levulinate, calcium oleate, calcium monobasic or tribasic phosphate, calcium pidolate, calcium sulfate and mixtures thereof. Preferably, the calcium is provided as calcium glycinate, calcium bisglycinate, calcium hydroxide or mixtures thereof; preferably calcium carbonate, calcium phosphate and calcium citrate; more preferably calcium carbonate or calcium hydrogen phosphate; more preferably calcium carbonate comprising a 40 wt % of elemental calcium of the total weight of calcium carbonate, calcium hydrogen phosphate comprising 29.5 wt % of elemental calcium and/or mixtures thereof. In a particular embodiment, the capsule filing composition comprises calcium carbonate (40% Ca) in an amount between 50 and 700 mg, preferably between 100 and 500 mg, and/or calcium hydrogen phosphate (29.5% Ca) in an amount between 100 and 1000 mg, preferably between 150 and 750 mg.

In a more particular embodiment, the capsule filling composition further comprises magnesium, preferably in an amount between 1 and 10 wt % of the total weight of the soft gelatin capsule; more preferably in between 3 and 9 wt %. In a preferred embodiment, the magnesium is in an amount between 10 and 100 mg; preferably between 35 and 75 mg.

In a particular embodiment the capsule filling composition further comprises at least a compound comprising magnesium. Non-limiting examples of suitable compounds comprising magnesium are magnesium oxide, magnesium citrate, magnesium chloride, magnesium acetate; magnesium glycinate and combinations thereof; preferably magnesium oxide, magnesium citrate, magnesium chloride and magnesium acetate; more preferably magnesium oxide; even more preferably magnesium oxide heavy. In a particular embodiment the capsule filing composition comprises magnesium oxide heavy (60% Mg) in an amount between 10 and 300 mg, preferably between 50 and 150 mg.

In a more particular embodiment, the capsule filling composition further comprises manganese, preferably in an amount between 0.01 and 1.00 wt % of the total weight of the soft gelatin capsule; more preferably in between 0.05 and 0.50 wt %. In a preferred embodiment, the manganese is in an amount between 0.5 and 5 mg; preferably about 2 mg.

In a particular embodiment the capsule filling composition further comprises at least a compound comprising manganese. Non-limiting examples of suitable compounds comprising manganese are manganese gluconate, manganese sulfate, manganese ascorbate, amino acid chelates of manganese and combinations thereof; preferably manganese sulfate; more preferably manganese (II) sulfate hydrate.

In a particular embodiment, the capsule filling composition further comprises: selenium and/or iodine and, optionally, calcium.

In a particular embodiment, the capsule filling composition further comprises:
a compound comprising selenium;
a compound comprising iodine; and
optionally, a compound comprising calcium.

In a particular embodiment, the capsule filling composition further comprises:
between 10 and 200 μg of sodium selenite;
between 50 and 500 μg of potassium iodate; and
optionally, between 50 and 700 mg of calcium carbonate.

In an even more particular embodiment, the capsule filling composition further comprises:
between 20 and 150 μg of sodium selenite;
between 100 and 400 μg of potassium iodate; and
optionally, between 100 and 500 mg of calcium carbonate.

### Vitamins

The capsule filling composition comprises at least a humidity sensitive vitamin.

In the context of the present invention, a “humidity sensitive vitamin” is understood as a vitamin that may be degraded and/or that loses biological activity when exposed to humid, damp or moist conditions, such as upon exposure to a relative humidity of 50%, such as during one month, such as at or near ordinary room temperature (between 20 and 35° C.). In particular, the humidity sensitive vitamin may be degraded or lose activity in the presence of a hygroscopic compound [i.e. a compound that can absorb or adsorb water from its surroundings]. In a particular embodiment the hygroscopic compound is choline glycerophosphate (GPC). Moreover, the presence of water adsorbed by the GPC may contribute to degradation of vitamins either directly or by facilitating interactions with other components.

In a particular embodiment, a “humidity sensitive vitamin” is a vitamin that degrades in at least a 0.5 wt %, at least 1 wt %, at least 2 wt %, at least 3 wt %, at least 5 wt %, at least 10 wt %, at least 20% or at least 30 wt % of the total initial weight of the vitamin, preferably after 1, 2 3 or 4 months of being stored at room temperature (for example between 20 and 35° C.) and at a relative humidity of between 10 and 60%; more preferably after 4 months of being stored in an environmentally controlled storage room at between 30 and 31° C. and at a relative humidity of between 12 and 58%; particularly at about 30.8° C.

In a particular embodiment, the vitamin is stored in the dark.

In a particular embodiment, a “humidity sensitive vitamin” is a vitamin that degrades in at least a 0.5 wt %, at least 1 wt %, at least 2 wt % or at least 3 wt % of the total initial weight of the vitamin, preferably after at least 3 months of being stored at a temperature of at least 25° C. and at a relative humidity of at least 60%.

In a particular embodiment, a “humidity sensitive vitamin” is a vitamin that degrades in at least a 5 wt % of the total initial weight of the vitamin, preferably after at least 3 months of being stored at a temperature of at least 25° C. and at a relative humidity of at least 60%.

In a particular embodiment, a “humidity sensitive vitamin” is a vitamin that degrades in at least a 10 wt % of the total initial weight of the vitamin, preferably after at least 3 months of being stored at a temperature of at least 25° C. and at a relative humidity of at least 60%.

In a particular embodiment, a “humidity sensitive vitamin” is a vitamin that degrades in at least a 20 wt % of the total initial weight of the vitamin, preferably after at least 3 months of being stored at a temperature of at least 25° C. and at a relative humidity of at least 60%.

In a particular embodiment, the at least a humidity sensitive vitamin is selected from the group consisting of vitamin A, vitamin C, vitamin D, vitamin B1, vitamin B2, vitamin B5, vitamin B6, vitamin B9, vitamin B12, vitamin K and combinations thereof; preferably vitamin A, vitamin C, vitamin D, vitamin B1, vitamin B2, vitamin B5, vitamin B6, vitamin B9, vitamin B12 and combinations thereof; preferably is selected from the group consisting of vitamin A, vitamin D, vitamin B2, vitamin B5, vitamin B6, vitamin B12 and combinations thereof.

In another particular embodiment, the at least a humidity sensitive vitamin is selected from the group consisting of vitamin C, vitamin B1, vitamin B5, vitamin B12 and combinations thereof.

Non-limiting examples of suitable vitamins for the capsule filling composition of the present invention are vitamin A, vitamin C, vitamin D, vitamin E, vitamin K, vitamin B such as thiamine (B1), riboflavin (B2), niacin (B3), pantothenic acid (B5), pyroxidine (B6), biotin (B8), folate (B9), cobalamin (B12), and mixtures thereof; preferably vitamin A, vitamin B1, vitamin B2, Vitamin B3, vitamin B5, vitamin B6, vitamin B8, vitamin B9, vitamin B12, vitamin C, vitamin D3, vitamin E and/or mixtures thereof.

In an embodiment, the capsule filling composition further comprises vitamin A or provitamin A, preferably β carotene, in an amount between 0.005 and 0.200 wt % of the total weight of the soft gelatin capsule; preferably between 0.01 and 0.10 wt %; more preferably comprises vitamin A in an amount between 100 and 1500 μg RE (expressed as retinol equivalents, RE); particularly between 100 and 1000 μg RE; preferably between 200 and 1000 μg RE; particularly between 200 and 800 μg RE. Particularly, the capsule filling composition comprises vitamin A (1 MM palmitate in an amount between 0.5 mg and 5 mg; preferably between 1 and 3 mg.

In the context of the present invention the term “vitamin A” is as known in the art and also refers to retinol, retinaldehyde, retinoic acid, retinoids, retinal and/or retinoic ester, and provitamin A also refers to β-carotene, α-carotene and β-cryptoxanthin. The capsule filling composition may comprise Vitamin A in form of retinyl esters such as retinyl palmitate, retinyl oleate, retinyl stearate, retinyl linoleate or combination thereof.

In the context of the present invention one retinol equivalent (RE) is defined as the biological activity associated with 1 μg of all-trans retinol as known in the art, for example, 1333 UI of vitamin A are 400 micrograms of RE.

In an embodiment, the capsule filling composition further comprises at least a vitamin of the vitamin B complex. In a more particular embodiment, vitamin B complex comprises a mixture of vitamin B1 (thiamin), vitamin B2 (riboflavin or vitamin G), Vitamin B3 (niacin, nicontinin acid or vitamin PP), vitamin B5 (pantothenic acid), vitamin B6 (pyridoxine, pyridoxal or pyridoxamine), vitamin B8 (biotin or vitamin H), vitamin B9 (folic acid, folate, folacin, pteroyl-L-glitamic acid, or vitamin M) and vitamin B12 (cobalamin or cyanocombalamin).

In an embodiment, the capsule filling composition further comprises vitamin B1 in an amount between 0.005 and 1.000 wt % of the total weight of the soft gelatin capsule; preferably between 0.001 and 0.50 wt %; more preferably in an amount between 0.05 and 3 mg; particularly between 0.1 and 3 mg; more particularly between 0.2 and 2.5 mg; preferably between 0.1 and 2 mg.

In the context of the present invention the term “vitamin B1” is as known in the art and also refers to thiamine and its salts (for example, thiamine mononitrate)

In an embodiment, the capsule filling composition further comprises vitamin B2 in an amount between 0.005 and 1.000 wt % of the total weight of the soft gelatin capsule; preferably between 0.001 and 0.500 wt %; more preferably in an amount between 0.30 and 5 mg; preferably between 0.5 and 3 mg.

In the context of the present invention the term “vitamin B2” is as known in the art and also refers to riboflavin.

In an embodiment, the capsule filling composition further comprises vitamin B3 in an amount between 0.05 and 5.00 wt % of the total weight of the soft gelatin capsule; preferably between 0.30 and 3.00 wt %; more preferably in an amount between 1 and 50 mg, particularly between 5 and 35 mg; preferably between 5 and 30 mg. In the context of the present invention the term “vitamin B3” is as known in the art and also refers to vitamin PP, niacin, nicotinic acid and its derivatives (for example, nicotinamide or niacinamide).

In an embodiment, the capsule filling composition further comprises vitamin B5 in an amount between 0.01 and 2.00 wt % of the total weight of the soft gelatin capsule; preferably between 0.10 and 1.50 wt %; more preferably in an amount between 1 and 15 mg, particularly between 1.5 and 10 mg; preferably between 3 and 10 mg. In the context of the present invention the term “vitamin B5” is as known in the art and also refers to vitamin W, pantothenic acid and its derivatives (for example, D-Calcium pantothenate)

In an embodiment, the capsule filling composition further comprises vitamin B6 in an amount between 0.005 and 1.000 wt % of the total weight of the soft gelatin capsule; preferably between 0.001 and 0.50 wt %; more preferably in an amount between 0.1 and 5 mg; preferably between 0.5 and 3 mg; more preferably between 0.5 and 3.5 mg.

In the context of the present invention the term “vitamin B6” is as known in the art and also refers to thiamine and its salts (thiamine mononitrate).

In an embodiment, the capsule filling composition further comprises vitamin B8 in an amount between 0.0005 and 0.10 wt % of the total weight of the soft gelatin capsule; preferably between 0.001 and 0.05 wt %; more preferably in an amount between 10 and 100 μg; preferably between 20 and 80 μg.

In the context of the present invention the term “vitamin B8” is as known in the art and also refers to vitamin H or biotine.

In an embodiment, the capsule filling composition further comprises folic acid (vitamin B9) or a glucosamine salt of (6S)-5-methyltetrahydrofolate (Quatrefolic®). Preferably it comprises folic acid in an amount between 0.001 and 0.100 wt % of the total weight of the soft gelatin capsule; more preferably between 0.0005 and 0.05 wt %; even more preferably in an amount of between 0.01 and 1.00 mg; preferably between 0.05 and 1.00 mg; more preferably between 0.05 and 0.70 mg; even more preferably between 0.1 and 0.7 mg; or preferably a glucosamine salt of (6S)-5-methyl tetrahydrofolate between 0.01 and 0.10 wt % of the total weight of the soft gelatin capsule; even more preferably in an amount of between 0.05 and 2.00 mg; preferably between 0.10 and 1.00 mg.

In an embodiment, the capsule filling composition further comprises vitamin B12 in an amount between 0.0001 and 0.010 wt % of the total weight of the soft gelatin capsule; more preferably in an amount between 0.5 and 10 μg; preferably between 1 and 7 μg; more preferably between 1 and 5 μg.

In the context of the present invention the term “vitamin B8” is as known in the art and also refers to cobalamin.

In an embodiment, the capsule filling composition further comprises vitamin C; preferably in an amount between 0.10 and 10 wt % of the total weight of the soft gelatin capsule; preferably between 1.00 and 5.00 wt %; even more preferably in an amount between 10 and 300 mg; particularly between 10 and 200 mg; preferably between 20 and 200 mg; more preferably between 20 and 100 mg.

In the context of the present invention the term “vitamin C” is as known in the art and also refers to ascorbic acid. The capsule filling composition may comprise Vitamin C in form of ascorbic acid, sodium ascorbate, calcium ascorbate, ascorbic palmitate or in a combination thereof.

In an embodiment, the capsule filling composition further comprises vitamin D; preferably in an amount between 0.0001 and 0.0100 wt % of the total weight of the soft gelatin capsule.

In the context of the present invention the term “vitamin D” might be selected from D1 to D5 vitamins and thus refers to calciferol, cholecalciferol, lumisterol, ergocalciferol, dihydrotachysterol, 7-dehydrocholesterol and/or combinations thereof. In a more particular embodiment, vitamin D is vitamin D3 (cholecalciferol); preferably in an amount between 0.0005 and 0.005 wt % of the total weight of the soft gelatin capsule, even more preferably in an amount between 0.5 and 50 μg; preferably between 1 and 35 μg; more preferably between 1 and 30 μg.

In an embodiment, the capsule filling composition further comprises vitamin E; preferably in an amount between 0.10 and 5.00 wt % of the total weight of the soft gelatin capsule. In the context of the present invention the term “vitamin E” is as known in the art and comprises four tocopherols and four tocotrienols, each one in alpha, beta, gamma and delta form. In a more particular embodiment, vitamin E is alfa tocopherol; preferably alfa tocopherol acetate; preferably between 0.20 and 2.00 wt %; even more preferably in an amount between 1 and 30 mg; preferably between 5 and 15 mg.

In an embodiment, the capsule filling composition further comprises a mixture of vitamins; preferably wherein the mixture of vitamins is in an amount between 0.01 and 5 wt % of the total weight of the soft gelatin capsule; preferably between 0.02 and 2 wt %.

In another particular embodiment, the capsule filling composition further comprises a mixture of vitamin B1, vitamin B2, Vitamin B3, vitamin B5, vitamin B6, vitamin B8, vitamin B9, vitamin B12, and vitamin D3 and, optionally, vitamin A and/or vitamin C.

In a more particular embodiment, the capsule filling composition further comprises a vitamin selected from the group consisting of vitamin A, vitamin C, vitamin D, vitamin B1, vitamin B2, vitamin B5, vitamin B6, vitamin B9, vitamin B12, vitamin K and combinations thereof; preferably of vitamin A, vitamin C, vitamin D, vitamin B1, vitamin B2, vitamin B5, vitamin B6, vitamin B9, vitamin B12, and combinations thereof; more preferably vitamin A, vitamin D, vitamin B2, vitamin B5, vitamin B6, vitamin B12 and combinations thereof.

In another embodiment, the capsule filling composition further comprises a vitamin selected from the group consisting of vitamin B1, vitamin B5, vitamin B12, vitamin C, and combinations thereof.

In a particular embodiment, the capsule filling composition further comprises a mixture of vitamin B1, vitamin B2, Vitamin B3, vitamin B5, vitamin B6, vitamin B8, vitamin B9, vitamin B12, vitamin D3 and, optionally, vitamin C.

In a particular embodiment the capsule filling composition further comprises a mixture of vitamin A, vitamin B1, vitamin B2, Vitamin B3, vitamin B5, vitamin B6, vitamin B8, vitamin B9, vitamin B12, vitamin D3, vitamin E and, optionally, vitamin C.

In an even more particular embodiment, the capsule filling composition comprises:
between 50 and 1000 mg of choline glycerophosphate;
between 100 and 1500 mg of fish oil comprising eicosapentaenoic acid (EPA) and docosahexaenoic acid (DHA); wherein the weight ratio of EPA:DHA is between 1:50 and 1:1.2;
between 15 and 150 mg of ferrous fumarate or between 1 and 150 mg of ferrous bisglycinate chelate;
between 1 and 30 mg of zinc oxide;
optionally, between 0.5 and 5 mg of anhydrous copper sulfate; particularly anhydrous copper sulfate or copper sulfate 1-hydrate;
between 10 and 200 μg of sodium selenite;
between 50 and 500 μg of potassium iodate;
optionally, 50 and 700 mg of calcium carbonate; and:
between 0.3 and 5 mg of vitamin B2; or between 1 and 15 mg of vitamin B5; or between 0.1 and 5 mg of vitamin B6; between 50 and 1000 μg of vitamin B9 or between 100 and 2000 μg of glucosamine salt of (6S)-5-methyltetrahydrofolate; or between 0.5 and 10 μg of vitamin B12; or between 1 and 30 μg of vitamin D3; and
optionally, between 100 and 1500 μg RE of vitamin A.

In an even more particular embodiment, the capsule filling composition comprises:
between 50 and 1000 mg of choline glycerophosphate;
between 100 and 1500 mg of fish oil comprising eicosapentaenoic acid (EPA) and docosahexaenoic acid (DHA); wherein the weight ratio of EPA:DHA is between 1:50 and 1:1.2;
between 15 and 150 mg of ferrous fumarate or between 1 and 150 mg of ferrous bisglycinate chelate;
between 1 and 30 mg of zinc oxide;
optionally, between 0.5 and 5 mg of copper sulfate; particularly anhydrous copper sulfate or copper sulfate 1-hydrate;
between 10 and 200 μg of sodium selenite;
between 50 and 500 μg of potassium iodate;
optionally, 50 and 700 mg of calcium carbonate; and
between 0.3 and 5 mg of vitamin B2; and/or
between 1 and 15 mg of vitamin B5; and/or
between 0.1 and 5 mg of vitamin B6; and/or
between 50 and 1000 μg of vitamin B9 or between 100 and 2000 μg of glucosamine salt of (6S)-5-methyltetrahydrofolate; and/or
between 0.5 and 10 μg of vitamin B12; and/or
between 1 and 30 μg of vitamin D3; and
optionally, between 100 and 1500 μg RE of vitamin A.

In an even more particular embodiment, the capsule filling composition further comprises:
between 0.05 and 3 mg of vitamin B1; and
optionally, between 10 and 300 mg of vitamin C.

In an even more particular embodiment, the capsule filling composition further comprises:
between 5 and 50 mg of vitamin B3; and/or
between 10 and 100 μg of vitamin B8; and/or
between 1 and 30 mg of vitamin E; and
optionally, taurine preferably between in an amount 10 and 50 mg.

In an even more particular embodiment, the capsule filling composition comprises:
between 50 and 900 mg of choline glycerophosphate;
between 100 and 1500 mg of fish oil comprising eicosapentaenoic acid (EPA) and docosahexaenoic acid (DHA); wherein the weight ratio of EPA:DHA is between 1:50 and 1:1.2;
between 10 and 150 mg of ferrous fumarate and/or between 1 and 150 mg of ferrous bisglycinate chelate;
optionally, between 0.5 and 5 mg of copper sulfate; particularly anhydrous copper sulfate or copper sulfate 1-hydrate;
between 0.1 and 3 mg of vitamin B1; and/or
between 1 and 15 mg of vitamin B5; and/or
between 0.5 and 10 μg of vitamin B12; and
optionally, between 10 and 200 mg of vitamin C.

In an even more particular embodiment, the capsule filling composition comprises:
between 50 and 900 mg of choline glycerophosphate;
between 100 and 1500 mg of fish oil comprising eicosapentaenoic acid (EPA) and docosahexaenoic acid (DHA); wherein the weight ratio of EPA:DHA is between 1:50 and 1:1.2;
between 10 and 150 mg of ferrous fumarate and/or between 1 and 150 mg of ferrous bisglycinate chelate;
optionally, between 0.5 and 5 mg of copper sulfate; particularly anhydrous copper sulfate or copper sulfate 1-hydrate
between 10 and 200 μg of sodium selenite; and
between 0.1 and 3 mg of vitamin B1; and/or
between 1 and 15 mg of vitamin B5; and/or
between 0.5 and 10 μg of vitamin B12; and
optionally, between 10 and 200 mg of vitamin C.

In an even more particular embodiment, the capsule filling composition further comprises:
between 0.3 and 5 mg of vitamin B2; and/or
between 0.1 and 5 mg of vitamin B6; and/or
between 1 and 50 μg of vitamin D3; and/or
between 10 and 1000 μg of vitamin B9; and/or
optionally, between 100 and 1000 μg RE of vitamin A.

In an even more particular embodiment, the capsule filling composition further comprises:
between 1 and 50 mg of vitamin B3; and/or
between 10 and 100 μg of vitamin B8; and/or
between 1 and 30 mg of vitamin E; and
optionally, taurine preferably between in an amount 10 and 50 mg.

In an even more particular embodiment, the capsule filling composition further comprises
between 0.1 and 3 mg of vitamin B1; and/or
between 0.3 and 5 mg of vitamin B2; and/or
between 1 and 50 mg of vitamin B3; and/or
between 1 and 15 mg of vitamin B5; and/or
between 0.1 and 5 mg of vitamin B6; and/or
between 10 and 100 μg of vitamin B8; and/or
between 0.05 and 1.0 mg of vitamin B9 or between 0.1 and 2.0 mg of glucosamine salt of (6S)-5-methyltetrahydrofolate; and/or
between 0.5 and 10 μg of vitamin B12; and/or
between 1 and 50 μg of vitamin D3; and/or
optionally, between 10 and 200 mg of vitamin C.

In an even more particular embodiment, the capsule filling composition further comprises
between 0.5 and 2 mg of vitamin B1;
between 1 and 2.5 mg of vitamin B2;
between 10 and 30 mg of vitamin B3;
between 5 and 10 mg of vitamin B5;
between 1 and 3 mg of vitamin B6;
between 30 and 60 μg of vitamin B8;
between 0.3 and 0.5 mg of vitamin B9 or between 0.5 and 1.0 mg of glucosamine salt of (6S)-5-methyltetrahydrofolate;
between 2 and 5 μg of vitamin B12;
between 10 and 30 μg of vitamin D3;
and, optionally, between 30 and 50 mg of vitamin C;

In an even more particular embodiment, the capsule filling composition further comprises
between 0.5 mg and 5 mg of vitamin A palmitate; and/or
between 0.1 and 3 mg of vitamin B1; and/or
between 0.3 and 5 mg of vitamin B2; and/or
between 1 and 50 mg of vitamin B3; and/or
between 1 and 15 mg of vitamin B5; and/or
between 0.1 and 5 mg of vitamin B6; and/or
between 10 and 100 μg of vitamin B8; and/or
between 0.01 and 0.5 mg of vitamin B9 or between 0.02 and 1.0 mg of glucosamine salt of (6S)-5-methyltetrahydrofolate; and/or
between 0.5 and 5 μg of vitamin B12; and/or
optionally, between 10 and 200 mg of vitamin C; and/or
between 0.5 and 30 μg of vitamin D3; and/or
between 1 and 30 mg of vitamin E.

In an even more particular embodiment, the capsule filling composition further comprises
between 1 and 3 mg of vitamin A palmitate;
between 0.3 and 1.5 mg of vitamin B1;
between 0.5 and 1.5 mg of vitamin B2;
between 5 and 15 mg of vitamin B3;
between 2 and 6 mg of vitamin B5;
between 0.5 and 1.5 mg of vitamin B6;
between 20 and 40 μg of vitamin B8;
between 0.05 and 0.3 mg of vitamin B9 or between 0.1 and 0.6 mg of glucosamine salt of (6S)-5-methyltetrahydrofolate;
between 1 and 3 μg of vitamin B12;
optionally, between 30 and 50 mg of vitamin C;
between 1 and 10 μg of vitamin D3; and
between 5 and 10 mg of vitamin E.

In an even more particular embodiment, the capsule filling composition comprises:
between 50 and 900 mg of choline glycerophosphate;
between 100 and 1500 mg of fish oil comprising eicosapentaenoic acid (EPA) and docosahexaenoic acid (DHA); wherein the weight ratio of EPA:DHA is between 1:50 and 1:1.2;
between 10 and 150 mg of ferrous fumarate and/or between 1 and 150 mg of ferrous bisglycinate chelate;
optionally, between 0.5 and 5 mg of copper sulfate;
between 0.1 and 3 mg of vitamin B1; or
between 1 and 15 mg of vitamin B5; or
between 0.5 and 10 μg of vitamin B12; and
optionally, between 10 and 200 mg of vitamin C.

In an even more particular embodiment, the capsule filling composition comprises:
between 50 and 900 mg of choline glycerophosphate;
between 100 and 1500 mg of fish oil comprising eicosapentaenoic acid (EPA) and docosahexaenoic acid (DHA); wherein the weight ratio of EPA:DHA is between 1:50 and 1:1.2;
between 10 and 150 mg of ferrous fumarate and/or between 1 and 150 mg of ferrous bisglycinate chelate;
optionally, between 0.5 and 5 mg of copper sulfate;
between 0.1 and 3 mg of vitamin B1; and/or
between 1 and 15 mg of vitamin B5; and/or
between 0.5 and 10 μg of vitamin B12; and
optionally, between 10 and 200 mg of vitamin C.

In an even more particular embodiment, the capsule filling composition further comprises:
between 0.3 and 5 mg of vitamin B2; and/or
between 0.1 and 5 mg of vitamin B6; and/or
between 10 and 1000 μg of vitamin B9; and/or
between 0.5 and 50 μg of vitamin D3; and
optionally, between 100 and 1000 μg RE of vitamin A.

In an even more particular embodiment, the capsule filling composition further comprises:
between 1 and 50 mg of vitamin B3; and/or
between 10 and 100 μg of vitamin B8; and/or
between 1 and 30 mg of vitamin E; and
optionally, between 10 and 50 mg of taurine.

In an even more particular embodiment, the capsule filling composition further comprises
between 0.05 and 3 mg of vitamin B1; and/or
between 0.3 and 5 mg of vitamin B2; and/or
between 5 and 50 mg of vitamin B3; and/or
between 1 and 15 mg of vitamin B5; and/or
between 0.1 and 5 mg of vitamin B6; and/or
between 10 and 100 μg of vitamin B8; and/or
between 0.05 and 1.0 mg of vitamin B9; and/or
between 0.5 and 10 μg of vitamin B12; and/or
between 1 and 30 μg of vitamin D3; and/or
optionally, between 10 and 300 mg of vitamin C;
and, optionally, 0.30 and 1.00 mg of provitamin A.

In an even more particular embodiment, the capsule filling composition further comprises:
between 0.5 and 1.5 mg of vitamin B1;
between 1 and 2 mg of vitamin B2;
between 10 and 20 mg of vitamin B3;
between 5 and 7 mg of vitamin B5;
between 1 and 2 mg of vitamin B6;
between 40 and 60 μg of vitamin B8;
between 0.3 and 0.5 mg of vitamin B9;
between 2 and 5 μg of vitamin B12;
between 10 and 20 μg of vitamin D3; optionally between 30 and 50 mg of vitamin C;
and, optionally, 0.50 and 0.70 mg of provitamin A

In an embodiment, the capsule filling composition further comprises vitamin K such as phylloquinone and/or naphthoquinone.

### Other Active Ingredients

In an embodiment, the capsule filling composition further comprises other compounds classified as vitamins by some authorities (or as pseudo-vitamins) such as termed and include, but are not limited to, ubiquinone (coenzyme QIO), pangamic acid, dimethylglycine, taestrile, amygdaline, flavanoids, para-aminobenzoic acid, adenine, adenylic acid, s-methylmethioninc and combinations thereof.

In an embodiment, the capsule filing composition further comprises taurine (2-aminoethanesulfonic acid); preferably in an amount between 0,1 and 10wt % of the total weight of the soft gelatin capsule; preferably in an amount between 0.5 and 5 wt %; more preferably between 10 and 50 mg; preferably between 15 and 35 mg.

### Excipients

The soft capsule filling composition may contain additional non-active ingredients, such us pharmaceutically acceptable excipients.

In a particular embodiment, the capsule filling composition further comprises wax; preferably bee wax, more preferably yellow beewax. In an embodiment, the wax is in between 0.01 and 10 wt % of the total weight of the soft gelatin capsule; preferably between 0.1 and 5 wt %; more preferably between 1 and 100 mg; even more preferably between 10 and 80 mg; even much more preferably between 20 and 70 mg; particularly between 20 and 60 mg.

The term “wax” is known in the art and refers a class of chemical compounds that are plastic (malleable) near room temperatures. Characteristically, they melt above 45° C. to give a low viscosity liquid. Waxes are insoluble in water but soluble in organic, nonpolar solvents. Waxes may contain esters of carboxylic acids and long chain alcohols or mixtures of substituted hydrocarbons, such as long chain fatty acids and primary alcohols (said substituted hydrocarbons having a hydrocarbon chain comprising more than 20 carbon atoms). Synthetic waxes are long-chain hydrocarbons lacking functional groups. Non-limiting examples of waxes suitable for the present invention are synthetic wax, microcrystalline wax, paraffin wax, carnauba wax, beeswax, Chinese wax (insect wax), schellac wax, (lac wax), whale spermaceti, lanolin (wool wax), ouricuri wax, candelilla wax, esparto wax, ozocerite, and Montan wax.

In a particular embodiment, the capsule filling composition further comprises a vegetable oil; preferably a flaxseed oil (also known as linseed oil) or sunflower oil;

more preferably flaxseed oil between 0.50 and 20.00 wt % of the total weight of the soft gelatin capsule; more preferably of between 1 and 10 wt %; even more preferably in an amount between 50 and 200 mg; even much more preferably about 80-100 mg.

In a particular embodiment, the capsule filling composition further comprises an emulsion enhancing agent (emulsifier) such as tricaprin, trilaurin, trimyrstin, tripalmitin, tristearin or a mixture thereof; preferably a mixture of acrylic, capric, myristic, and stearic triglycerides (such as Sofistan®); preferably between 0.05 and 10 wt % of the total weight of the soft gelatin capsule; particularly between 0.1 and 7 wt %; more preferably between 0.1 and 5 wt %; particularly between 5 and 100 mg; more particularly between 10 and 70 mg; preferably between 5 and 60 mg; even more preferably between 10 and 50 mg.

In another preferred embodiment, the capsule filling composition of the soft capsule comprises a surfactant. The term “surfactant” refers to compounds that are amphiphilic, i.e. they contain both hydrophobic groups (tail) and hydrophilic groups (head), therefore, a surfactant contains both a water insoluble (or oil soluble) component, i.e. tail, and a water soluble component, i.e. head. Examples of surfactants are lecithin, polyoxyethylene products of hydrogenated vegetable oils, polyoxyethylene-sorbitan-fatty acid esters, and the like, for example, polyoxyethylene (50) hydrogenated castor oil which is commercialized under the trade mark Nikkol® (Nikkol HCO-50), polyoxyethylene (40) hydrogenated castor oil (Nikkol HCO-40), polyoxyethylene (60) hydrogenated castor oil (Nikkol HCO-60); polyoxyethylene (20) sorbitan monolaurate (polysorbate 20) which is commercialized under the trade mark Tween® (Tween 20), polysorbate 21 (Tween 21), polysorbate 40 (Tween 40), polysorbate 60 (Tween 60), polysorbate 80 (Tween 80), polysorbate 81 (Tween 81); sorbitan monooleate (Span 80); polyoxy-35-castoir oil (Cremophor® EL); polyoxyethylated glycerides (Labrafil® M2125 Cs), polyoxyethylated oleic glycerides (Labrafil® M1944 Cs); caprylocaproyl polyoxyl-8-glycerides (Labrasol®); D-α-tocopheryl polyethylene glycol 1000 succinate (TPGS).

In a particular embodiment, the capsule filling composition further comprises lecithin; particularly in an amount between 0.05 and 8.00 wt % of the total weight of the soft gelatin capsule; preferably between 0.10 and 6.00 wt %; more preferably between 1.00 and 5.00 wt %; even more preferably between 2.00 and 4.00 wt %. Non-limiting examples of lecithin suitable as excipient for the present invention are egg, soy and sunflower lecithin; preferably sunflower lecithin in an amount less than 10 wt % of the total weight of the soft gelatin capsule; particularly between 10 and 100 mg; more particularly between 20 and 60 mg; preferably between 10 and 50 mg; more preferably between 15 and 45 mg.

In another preferred embodiment, the soft gelatin capsule has a hardness of between 5 and 20 N; preferably of between 8 and 15 N; more preferably of between 9 and 12 N. The hardness of the soft gelatin capsule is measured using a known test in the art. The soft capsules of the present invention are dried until reaching a hardness of between 5 and 20 N; preferably of between 8 and 15 N; more preferably of between 9 and 12 N.

In an embodiment, the capsule filling composition of the soft capsule of the present invention is a liquid or semi-solid composition (oily solution or suspension).

In an embodiment, the capsule filling composition of the soft capsule of the present invention comprises:
choline glycerophosphate in an amount between 50 and 1000 mg;
and
   fish oil comprising eicosapentaenoic acid (EPA) and docosahexaenoic acid (DHA) preferably, wherein the weight ratio of EPA:DHA is between 1:50 and 1:1.2, in an amount between 50 mg and 2000 mg;
   and
   at least a humidity sensitive vitamin selected from the group consisting of vitamin A, vitamin C, vitamin D such as D3, vitamin B1, vitamin B2, vitamin B5, vitamin B6, vitamin B9, vitamin B12 and combinations thereof; preferably selected from the group consisting of vitamin A, vitamin D3, vitamin B2, vitamin B5, vitamin B6, vitamin B12 and combinations thereof;
   and optionally at least a compound comprising an element selected from copper, iron, nickel, zinc, calcium, iodine, selenium and/or a combination thereof; preferably selected from the group consisting of copper, iron, zinc, iodine, selenium and combinations thereof.

In an embodiment, the capsule filling composition of the soft capsule of the present invention comprises:
between 70 and 700 mg of choline glycerophosphate;
between 100 and 1500 mg of fish oil comprising eicosapentaenoic acid (EPA) and docosahexaenoic acid (DHA); wherein the weight ratio of EPA:DHA is between 1:50 and 1:1.2;
between 15 and 150 mg of ferrous fumarate;
between 1 and 30 mg of zinc oxide;
optionally, between 0.5 and 5 mg of anhydrous copper sulfate;
between 10 and 200 μg of sodium selenite;
between 50 and 500 μg of potassium iodate;
optionally, 50 and 700 mg of calcium carbonate;
between 0.05 and 3 mg of vitamin B1; and/or
between 0.3 and 5 mg of vitamin B2; and/or
between 1 and 50 mg of vitamin B3; and/or
between 1 and 15 mg of vitamin B5; and/or
between 0.1 and 5 mg of vitamin B6; and/or
between 10 and 100 μg of vitamin B8; and/or
between 0.05 and 1mg of vitamin B9; and/or
between 0.5 and 10 μg of vitamin B12; and/or
between 1 and 30 μg of vitamin D3; and
optionally, between 100 and 1500 μg RE of vitamin A; and
optionally, between 10 and 300 mg of vitamin C; and/or
optionally, between 1 and 30 mg of vitamin E; and
   optionally, taurine preferably between in an amount 10 and 50 mg.

In an embodiment, the capsule filling composition of the soft capsule of the present invention comprises:
choline glycerophosphate in an amount between 50 and 900 mg; and
fish oil comprising eicosapentaenoic acid (EPA) and docosahexaenoic acid (DHA) with a weight ratio of EPA:DHA between 1:50 and 1:1.2, in an amount between 50 and 2000 mg;

and
at least a humidity sensitive vitamin selected from the group consisting of vitamin A, vitamin C, vitamin D3, vitamin B1, vitamin B2, vitamin B5, vitamin B6, vitamin B9, vitamin B12 and combinations thereof;

and, optionally,
at least a compound comprising an element selected from copper, iron, nickel, zinc, calcium, iodine, selenium and/or a combination thereof.

and, optionally,
taurine.

In an embodiment, the capsule filling composition of the soft capsule of the present invention comprises:
between 70 and 700 mg of choline glycerophosphate;
between 100 and 750 mg of fish oil comprising eicosapentaenoic acid (EPA) and docosahexaenoic acid (DHA); wherein the weight ratio of EPA:DHA is between 1:50 and 1:1.2;
between 10 and 150 mg of ferrous fumarate and/or between 1 and 150 mg of ferrous bisglycinate chelate;
optionally, between 0.5 and 5 mg of copper sulfate;
optionally, between 10 and 200 μg of sodium selenite;
between 0.1 and 3 mg of vitamin B1; and/or
between 1 and 15 mg of vitamin B5; and/or
between 0.5 and 10 μg of vitamin B12; and
optionally, between 10 and 200 mg of vitamin C

In an embodiment, the capsule filling composition of the soft capsule of the present invention comprises:
between 70 and 700 mg of choline glycerophosphate;
between 50 and 700 mg of DHA;
between 10 and 150 mg of ferrous fumarate or between 1 and 150 mg of ferrous bisglycinate chelate;
between 1 and 30 mg of zinc oxide;
optionally, between 0.5 and 5 mg of copper sulfate; preferably anhydrous copper sulfate or copper sulfate 1-hydrate;
between 10 and 200 μg of sodium selenite;
between 50 and 500 μg of potassium iodate;
optionally, 50 and 700 mg of calcium carbonate;
between 0.1 and 3 mg of vitamin B1; and/or
between 0.3 and 5 mg of vitamin B2; and/or
between 1 and 50 mg of vitamin B3; and/or
between 1 and 15 mg of vitamin B5; and/or
between 0.1 and 5 mg of vitamin B6; and/or
between 10 and 100 μg of vitamin B8; and/or
between 10 and 1000 μg of vitamin B9 and/or 50 and/or 2000 μg of glucosamine salt of (6S)-5-methyltetrahydrofolate; and/or
between 0.5 and 10 μg of vitamin B12; and/or
between 0.5 and 50 μg of vitamin D3; and
optionally, between 100 and 1000 μg RE of vitamin A; and
optionally, between 10 and 200 mg of vitamin C; and
optionally, between 1 and 30 mg of vitamin E; and
optionally, between 15 and 50 mg of taurine.

In a preferred embodiment, the capsule filling composition of the soft capsule of the present invention comprises:
between 100 and 600 mg of choline glycerophosphate;
between 100 and 500 mg of DHA;
between 15 and 100 mg of ferrous fumarate or between 10 and 100 mg of ferrous bisglycinate chelate;
between 2 and 20 mg of zinc oxide;
optionally, between 1 and 3 mg of copper sulfate;
between 20 and 150 μg of sodium selenite;
between 100 and 400 μg of potassium iodate;
between 0.2 and 2.5 mg of vitamin B1;
between 0.5 and 3 mg of vitamin B2;
between 5 and 35 mg of vitamin B3;
between 1.5 and 10 mg of vitamin B5;
between 0.5 and 3.5 mg of vitamin B6;
between 20 and 80 μg of vitamin B8;
between 50 and 700 μg of vitamin B9 or between 100 and 1000 μg of glucosamine salt of (6S)-5-methyltetrahydrofolate;
between 1 and 5 μg of vitamin B12;
between 1 and 35 μg of vitamin D3; and
optionally, between 200 and 800 μg RE of vitamin A; and
optionally, between 20 and 100 mg of vitamin C; and
optionally, between 5 and 15 mg of vitamin E; and
optionally, between 15 and 35 mg of taurine.

### Uses

Another aspect of the present invention is directed to the non-therapeutic use of the soft gelatin capsule as defined above in any of its particular embodiments as a food supplement; dietary supplement, health supplement or nutraceutical; particularly for a subject suffering or who could suffer from choline and/or DHA and/or vitamins insufficiency; preferably in a pregnant or lactating woman, a fetus, an infant or a child.

The term “subject” or “patient” typically includes humans. In a preferred embodiment, the subject is a male or woman human subject of any race or age. In a particular embodiment, the subject is a woman; particularly a pregnant or lactating woman, a fetus, an infant (i.e. child younger than one year of age) or a child (i.e. person between infancy and puberty).

As used herein, the terms “food supplement”, “dietary supplement”, and “health supplement”, refer to sources of nutrients or other substances with a nutritional or physiological effect whose purpose is to supplement the normal diet and to meet the requirements for individuals at risk of deficiencies. They are taken in small measured quantities and may be marketed ‘in dose’ form such as capsules.

In the EU the Food Supplement Directive 2002/46 EC, ‘food supplements’ mean foodstuffs with the purpose of supplement the normal diet and which are concentrated sources of nutrients or other substances with a nutritional or physiological effect, alone or in combination, marketed in dose form, namely forms such as capsules, pastilles, tablets, pills and other similar forms, sachets of powder, ampoules of liquids, drop dispensing bottles, and other similar forms of liquids and powders designed to be taken in measured small unit quantities.

In the US Dietary Supplement Health Education Act (1994), the term ‘dietary supplement’ means a product intended to supplement the diet that bears or contains one or more of the following dietary ingredients: a) a vitamin; b) a mineral; c) an herb or other botanical; d) an amino acid; e) a dietary substance for use by man to supplement the diet by increasing the total dietary intake; or a concentrate, metabolite, constituent, extract, or combination of any ingredient described in a), b), c), d), or e). It is intended for ingestion in pill, capsule, tablet, or liquid form and is not represented for use as a conventional food or as the sole item of a meal or diet.

According to the ASEAN Consultative Committee for Standards and Quality Traditional Medicine and Health Supplement Product Working Group (ACCSQ TMHS PWG), a “health supplement” means any product that is used to supplement a diet and to maintain, enhance and improve the healthy function of human body. It is presented in dosage forms to be administered in small unit doses such as capsules, tablets, powder, liquids and it shall not include any sterile preparations. In the context of the present invention, the term “nutraceutical” means a foodstuff, such as a fortified food or dietary supplement, that provides health benefits in addition to its basic nutritional value.

Another aspect of the present invention is directed to a method comprising the step of providing the soft gelatin capsule as defined above in any of its particular embodiments as a food supplement; dietary supplement, health supplement or nutraceutical.

### Medical Uses

In a further aspect, the present invention relates to a soft gelatin capsule as defined above in any of the particular embodiments for use in medicine, particularly in preventive medicine or preventive health.

A further aspect of the invention is directed to a soft gelatin capsule as defined above, for use in the treatment and/or prevention of a disease or pathology related to choline and/or DHA and/or vitamin insufficiency, particularly wherein the subject suffering or who could suffer from said disease or pathology is a pregnant woman, a lactating woman, a fetus, an infant or a child.

In an embodiment, the gelatin capsule as defined above is for use in the prevention of a birth defect or health, growth and/or development problem, disease or pathology related to choline insufficiency, particularly wherein the subject suffering or who can suffer from said problem, disease or pathology is a fetus, an infant or a child.

In a particular embodiment, the soft gelatin capsule is for use
in the prevention of neural tube defects and/or orofacial clefts and/or neurodevelopment problems, diseases or pathologies in a fetus; and/or
in the prevention of placental development problems disease or pathology in a pregnant woman; and/or
in the prevention of cognitive development problems, memory and learning functions problems, diseases or pathologies of infants and children.

In a more particular embodiment, the soft gelatin capsule is for use in the support of cognitive development, memory and learning functions; preferably visual memory, processing speed, attention and better cognitive performance; more preferably of infants and children.

In a particular embodiment, the soft gelatin capsule is for use in the prevention of preterm birth, low birthweight, perinatal death, neonatal intensive care and/or vision defects and brain development defects of fetus, infant and children.

In a particular embodiment, the soft gelatin capsule is for use in the treatment and/or prevention of liver damages such as nonalcoholic steatohepatitis (NASH) and nonalcoholic fatty liver disease (NAFLD or hepatosteatosis), muscle damages or any cardiovascular disease related to high homocysteine level.

A further aspect of the invention is directed to the use of soft gelatin capsule as defined above, in the manufacture of a medicament for the treatment and/or prevention of a disease or pathology related to choline and/or DHA and/or vitamin insufficiency, particularly wherein the subject suffering or who could suffer from said disease or pathology is a pregnant or lactating woman, an infant or a child.

In a further aspect, the invention is directed to a method of treatment and/or prevention of a disease or pathology related to choline and/or DHA and/or vitamin insufficiency, said method comprising administering to a patient in need of such a treatment or prevention a soft gelatin capsule as described above.

The term “treatment” or “to treat” in the context of this specification means administration of a compound or formulation according to the invention (i.e. a soft gelatin capsule) to ameliorate or eliminate the disease or one or more symptoms associated with said disease. “Treatment” also encompasses ameliorating or eliminating the physiological sequelae of the disease.

The term “ameliorate” in the context of this invention is understood as meaning any improvement on the situation of the patient treated.

The term “prevention” or “to prevent” refers to the reduction in the risk of acquiring or developing a given disease or disorder, or the reduction or inhibition of the recurrence or a disease or disorder.

The term “support” refers to the contribution of normal or improved evolution of the patient treated, avoiding o minimizing the risk of developing a given disease or disorder.

In particular, the soft gelatin capsule for use as defined above in any of the particular embodiments is for administration once every three months, once every two months, once a month, once every three weeks, once every two weeks, once a week, once a day, twice a day, three times a day or four times a day; preferably once or twice a day.

### Process

The soft capsules of the present invention may be prepared by any conventional manufacturing process known in the art for the preparation of soft capsules such as by a rotary-die process.

A further aspect of the present invention is directed to a process for preparing the soft gelatin capsule of the invention, said process comprising:
a) preparing a capsule filling composition comprising
   between 50 and 1000 mg choline glycerophosphate,
   at least an essential unsaturated fatty acid; and
   at least a humidity sensitive vitamin;
b) forming a soft gelatin capsule shell;
c) filling the soft gelatin capsule shell with the capsule filling composition of step (a) to obtain a soft gelatin capsule, and
d) drying the soft gelatin capsule obtained in step (c).

The capsule filling composition of step (a) of the process for preparing a soft gelatin capsule, is (i.e. having the same composition) the capsule filling composition as described in any of the embodiments of the present invention.

The soft gelatin capsule shell of step (b) of the process for preparing a soft gelatin capsule, is (i.e. having the same composition) the soft gelatin capsule shell as described in any of the embodiments of the present invention.

Steps (a) can be performed before step (b), step (b) can be performed before step (a), or steps (a) and (b) can be performed simultaneously.

Preferably, steps (b) and (c) are carried out simultaneously, for example by use of rotary die technology.

In a particular embodiment, step (a), (b), (c) and/or step (d) are carried out under controlled relative humidity (RH) conditions, such as 15% to 35% RH, preferably 20 to 30% RH, more preferably around 25% RH.

In an optional embodiment, step (d) comprises drying the capsule obtained in the previous step, for example in a rotary oven, tumble dryer and tunnel dryer. In a particular embodiment, step (d) is carried out under controlled relative humidity (RH) conditions, such as 10% to 30% RH, preferably 15% to 25% RH, more preferably 22% RH.

In an optional embodiment, step (d) comprises drying the capsule obtained in the previous step (c) to reach a hardness of between 5 and 20 N; preferably of between 8 and 15 N; more preferably of between 9 and 12 N. The hardness of the soft gelatin capsule is measured using a known test in the art.

A further optional aspect of the present invention is directed to a soft gelatin capsule obtainable by the process of the invention in any of its particular embodiments. All the characteristics described above for the soft gelatin capsule in any of its particular embodiments may be applied to the soft gelatin capsule obtainable by the process of the invention.

### EXAMPLES

The invention is illustrated by means of the following examples which in no case limit the scope of the invention.

### Example 1

### Soft Capsules

A) Soft gelatin capsules A comprising choline glycerophosphate were prepared. Once prepared, the capsules comprised a soft gelatin capsule shell 1 and a filling composition 1 as follows.

The soft gelatin capsule shell 1 of the capsules had the composition described in Table 1:

Soft gelatin capsule shell

| | | |
|---|---|---|
| Gelatin | 265.102 | mg |
| Glycerin | 101.775 | mg |
| E-172 (iron oxide, red) | 2.639 | mg |
| E-172 (iron oxide, black) | 0.676 | mg |

The filling composition 1 of the capsules had the composition described in the following table:

| | | |
|---|---|---|
| Fish oil 10EPA 50DHA TG (Omega 3-H ™ | 400.000 | mg |
| Fish oil from Huatai Biopharm Inc.) | | |
| Choline glycerophosphate (GPC) (85 wt %) | 239.650 | mg |
| Ferrous fumarate (32 wt % Fe) | 87.500 | mg |
| Zinc oxide (78 wt % Zn) | 12.820 | mg |
| Vitamin C | 46.000 | mg |
| Vitamin B1 (86.29 wt %) | 1.400 | mg |
| Vitamin B2 (100 wt %) | 1.540 | mg |
| Vitamin B3 | 16.000 | mg |
| Vitamin B5 (92 wt %) | 6.520 | mg |
| Vitamin B6 (81.9 wt %) | 1.710 | mg |
| Vitamin B8 (100 wt %) | 0.050 | mg |
| Vitamin B9 (91 wt %) | 0.480 | mg |
| Vitamin B12 (0.1 wt %) | 2.500 | μg |
| Vitamin D3 (1000000 IU oily vitamin D3/g) | 0.460 | mg |
| Potasium Iodate (59.88 wt % I, 40.12 wt % K) | 0.334 | mg |
| Sodium selenite (45 wt % Se) | 0.123 | mg |
| Bee wax | 36.000 | mg |
| Sunflower lecithin | 30.000 | mg |
| Hydrogenated Coco-Glycerides (Softisan ®) | 18.000 | mg |

B) Soft gelatin capsules B comprising choline glycerophosphate were prepared. Once prepared, the capsules comprised a soft gelatin capsule shell 2 and a filling composition 2 as follows.

The soft gelatin capsule shell 2 of the capsules had the composition described in Table 2:

Soft gelatin capsule shell

| | | |
|---|---|---|
| Gelatin | 134.054 | mg |
| Glycerin | 36.865 | mg |
| Sorbitol | 20.108 | mg |
| E-172 (iron oxide, red) | 1.247 | mg |
| E-172 (iron oxide, black) | 0.318 | mg |

The filling composition 2 of the capsules had the composition described in the following table:

| | | |
|---|---|---|
| Fish oil 10EPA 50DHA TG (Omega 3-H ™ | 200.000 | mg |
| Fish oil from Huatai Biopharm Inc.) | | |
| Choline glycerophosphate (GPC) (85%) | 119.830 | mg |
| Ferrous fumarate (32% Fe) | 21.870 | mg |
| Zinc oxide (78% Zn) | 6.410 | mg |
| Copper(II) sulfate anhydrous | 1.250 | mg |
| Sodium selenite (45% weight percentage of Se) | 0.022 | mg |
| Vitamin C | 46.000 | mg |
| Vitamin B3 | 8.000 | mg |
| Vitamin B5 (92% weight percentage) | 3.260 | mg |
| Vitamin B12 (0.1% weight percentage) | 1.250 | mg |
| Vitamin B6 (81.9% weight percentage) | 0.855 | mg |
| Vitamin B2 (100% weight percentage) | 0.770 | mg |
| Vitamin B1 (86.29% weight percentage) | 0.700 | mg |
| Vitamin B8 (100% weight percentage) | 0.025 | mg |
| Vitamin B9 (91% weight percentage) | 0.120 | mg |
| Vitamin D3 (1000000 IU oleosa | 0.115 | mg |
| VITAMIN D3/g) | | |
| Vitamin E (DL a-tocopherol) | 10.350 | mg |
| Vitamin A | 1.529 | mg |
| Potasium Iodate (59.88% I, 40.12% K | 0.167 | mg |
| weight percentage) | | |
| Calcium carbonate | 250.000 | mg |
| Taurine | 20.000 | mg |
| Bee wax | 1.700 | mg |
| Sunflower lecithin | 10.00 | mg |
| Hydrogenated Coco-Glycerides (Softisan) | 3.300 | mg |
| Linseed oil | 86.525 | mg |

C) Soft gelatin capsules C comprising choline glycerophosphate were prepared. Once prepared, the capsules comprised a soft gelatin capsule shell 3 and a filling composition 3 as follows.

The soft gelatin capsule shell 3 of the capsules had the composition described in Table 3:

Soft gelatin capsule shell

| | | |
|---|---|---|
| Gelatin | 245.096 | mg |
| Glycerin | 94.841 | mg |
| E-172 (iron oxide, red) | 2.307 | mg |
| E-172 (iron oxide, black) | 0.591 | mg |

The filling composition 3 of the capsules had the composition described in the following Table:

| | | |
|---|---|---|
| Fish oil 10EPA 50DHA TG (Omega 3-H ™ | 200.000 | mg |
| Fish oil from Huatai Biopharm Inc.) | | |
| Choline glycerophosphate (GPC) (85%) | 145.25 | mg |
| Ferrous fumarate (32% Fe) | 21.870 | mg |
| Zinc oxide (78% Zn) | 6.410 | mg |
| Copper(II) sulfate | 1.250 | mg |
| Sodium selenite (45 wt % Se) | 0.022 | mg |
| Vitamin C (ascorbic acid 100 wt %) | 46.000 | mg |
| Vitamin B3 (nicotinamide 100 wt %) | 8.000 | mg |
| Vitamin B5 (D-calcium pantothenate 92 wt %) | 3.260 | mg |
| Vitamin B12 (cyanocobalamin 0.1 wt % | 1.250 | mg |
| maltodextrin) | | |
| Vitamin B6 (pyridoxine hydrochloride 81.9 wt %) | 0.855 | mg |
| Vitamin B2 (riboflavin 100 wt %) | 0.770 | mg |
| Vitamin B1 (thiamine mononitrate 86.29 wt %) | 0.700 | mg |
| Vitamin B8 (biotin 100 wt %) | 0.025 | mg |
| Vitamin B9 (folic acid 91 wt %) | 0.120 | mg |
| Vitamin D3 (1000000 IU oily vitamin D3/g) | 0.115 | mg |
| Vitamin E (DL a-tocopherol acetate 67 wt %) | 10.350 | mg |
| Vitamin A (1 MM UI palmitate) | 1.529 | mg |
| Potasium Iodate (59.88 wt % I, 18.27 wt % K) | 0.167 | mg |
| Taurine | 20.000 | mg |
| Bee wax | 25.000 | mg |
| Sunflower lecithin | 48.000 | mg |
| Hydrogenated Coco-Glycerides (Softisan) | 50.000 | mg |
| Linseed oil | 86.525 | mg |

### Soft Capsule Preparation

The soft capsules were prepared as follows. First, a mixture of the filling composition 1 or 2 of Example 1 was prepared. Then, a soft gelatin capsule shell was formed and the shell was filled with the filling composition mixture by a rotary die process known in the art. The soft gelatin capsules formed were then (optionally) dried on a rotary oven, either in air or under vacuum, to equilibrium moisture content to reach a hardness of between 9-12 N with forced conditioned air of 20%-30% relative humidity at 21° C.-24° C. The drying technique may proceed with an infrared drying step to speed up the process.

### Example 2

### Stability Study of Soft Capsules Comprising Choline Glycerophosphate (GPC)

The stability of the soft gelatin capsules described in Example 1 (composition 1) packaged within an aluminium (20 microns) and PVC/PVDC 250/60 microns blister packaging was tested by keeping the capsules in an accelerated conditions chamber (40° C./75% Relative humidity (RH)) and long-term conditions chambers (25° C./60% RH and 30°/65% RH) for several months. The number of months under accelerated condition storage is expresses as T1, T2, etc. For example, 3 months of storage under accelerated conditions is expressed as T3.

Condition: 40° C./75% relative humidity (RH)

| Parameter | Specification | | T0 | T3 | T6 |
|---|---|---|---|---|---|
| Color | AP09_27 | | OK | OK | OK |
| Odor | Characteristic | | OK | OK | OK |
| Aspect | Soft capsule 16 oblong | | OK | OK | OK |
| Disintegration | ≤30 | min | 28 min | 28 min | 29 min |
| time | | | | | |
| Averaged weight | 1271.279 | mg ± 15% | OK | OK | OK |
| per capsule | | | | | |
| Peroxides index | <5 | mEq/kg O₂ | OK | OK | OK |

| Condition: 25° C./60% relative humidity (RH) | | | | | | | | |
|---|---|---|---|---|---|---|---|---|
| Parameter | Specification | T0 | T3 | T6 | T9 | T12 | T18 | T24 |
| Color | AP09_27 | OK | OK | OK | OK | OK | OK | OK |
| Odor | Characteristic | OK | OK | OK | OK | OK | OK | OK |
| Aspect | Soft capsule | OK | OK | OK | OK | OK | OK | OK |
| | 16 oblong | | | | | | | |
| Disintegration time | ≤30 min | 28 | 28 | 28 | 28 | 28 | 20 | 29 |
| | | min | min | min | min | min | min | min |
| Averaged weight | 1271.279 | OK | OK | OK | OK | OK | OK | OK |
| per capsule | mg ± 15% | | | | | | | |
| Peroxides index | <5 mEq/kg O₂ | OK | OK | OK | OK | OK | OK | OK |

| Condition: 30° C./65% relative humidity (RH) | | | | | | | | |
|---|---|---|---|---|---|---|---|---|
| Parameter | Specification | T0 | T3 | T6 | T9 | T12 | T18 | T24 |
| Color | AP09_27 | OK | OK | OK | OK | OK | OK | OK |
| Odor | Characteristic | OK | OK | OK | OK | OK | OK | OK |
| Aspect | Soft capsule | OK | OK | OK | OK | OK | OK | OK |
| | 16 oblong | | | | | | | |
| Disintegration time | ≤30 min | 28 | 28 | 28 | 28 | 28 | 25 | 29 |
| | | min | min | min | min | min | min | min |
| Averaged weight | 1271.279 | OK | OK | OK | OK | OK | OK | OK |
| per capsule | mg ± 15% | | | | | | | |
| Peroxides index | <5 mEq/kg O₂ | OK | OK | OK | OK | OK | OK | OK |

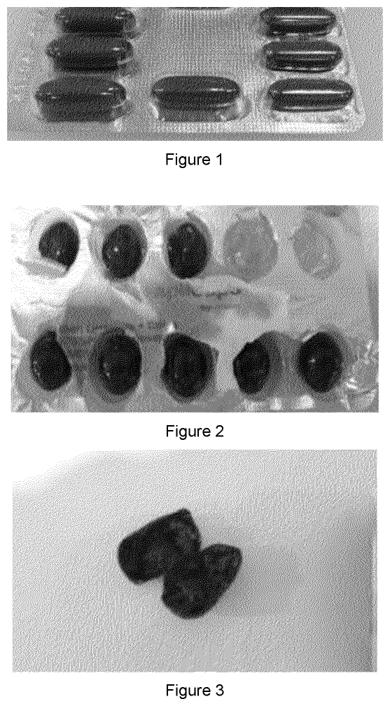

Results showed a good stability of the soft gelatin capsules up to 24 months (T24) in the different storage conditions studied. FIG. 1 shows a blister comprising soft gelatin capsules after 24 months at 25° C. and 60% of RH.

### Example 3

### Stability Study of Soft Capsules Comprising Choline Bitartrate

Soft gelatin capsules were prepared comprising the soft gelatin capsule shell and composition 1 as they have been described in Experiment 1, however in the present experiment choline glycerophosphate (GPC) was substituted by the same amount of choline bitartrate.

The stability of the soft gelatin capsules packaged within an aluminium (20 microns) and PVC/PVDC 250/60 microns blister packaging was tested after keeping the capsules in an accelerated conditions chamber (40° C./75% RH) and long-term conditions (25° C./60% RH and 30°/65% RH).

Results showed that after just three months (T3) under storage, several soft capsules were degraded. In particular, it was observed that after a swelling process, several capsules burst (were broken) loosing part of the filing. In addition, the color of the capsules turned to a darker shade (see FIGS. 2 and 3).

Thus, results showed that soft gelatin capsules comprising choline bitartrate were unstable.

### Example 4

### Stability Study of Soft Gelatin Capsules Comprising Choline Bitartrate without Metal Elements in the Filling

Soft gelatin capsules comprising choline bitartrate were prepared as in Example 3 but without metal compounds in the filling composition. In particular, ferrous fumarate, zinc oxide, sodium selenite and were not present in the filling composition of composition 1.

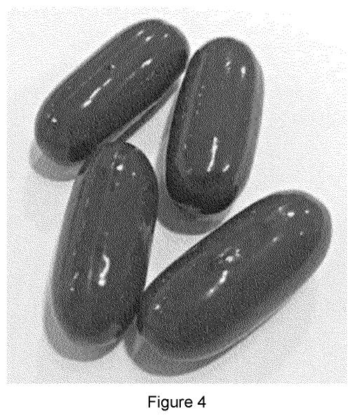

The stability of the soft gelatin capsules packaged within an aluminum (20 microns) and PVC/PVDC 250/60 microns blister was tested after keeping the packaged soft capsules in an accelerated conditions chamber (40° C./75% RH) and long-term conditions (25° C./60% RH and 30°/65% RH) up to 9 months (T9). Results showed that the capsules comprising choline bitartrate did not last three months stored at in an accelerated conditions chamber at 40° C./75% RH. In addition, after nine months stored (25° C./60% RH and 30°/65% RH), the soft gelatin capsules showed changes in their aspect (shape) and color, showing darker areas on the shell surface (see FIG. 4).

Thus, results showed that soft gelatin capsules comprising choline bitartrate were unstable even if compounds comprising metal elements are removed from the filling composition.

### Example 5

### Stability Study of Vitamins in Soft Capsules Comprising Choline Glycerophosphate (GPC)

In the present Example, the stability over time of several compounds of soft gelatin capsules was tested. Soft gelatin capsules comprising choline glycerophosphate were prepared as described in Example 1A.

Once prepared, the soft gelatin capsules were packaged within an aluminium (20 microns) and PVC/PVDC 250/60 microns blister packaging, the stability of the active compounds in those capsules was tested by keeping the capsules in an accelerated conditions chamber (40° C./75% Relative humidity (RH)) and long-term conditions chambers (25° C./60% RH and 30°/65% RH) for several months. The number of months “n” under accelerated condition storage is expressed as “Tn”; for example T0 refers to the original composition at the beginning of the test, and T3 to 3 months of storage under accelerated conditions. Notice that the amount of choline is obtained from choline glycerophosphate (GPC).

The filling composition of the capsules had the composition described in the following table after 0, 3 or 6 months storage in an accelerated conditions chamber (40° C./75% Relative humidity (RH)):

| | | | | |
|---|---|---|---|---|
| | Specification | | | |
| Parameter | per capsule | T0 | T3 | T6 |

| | | | | | |
|---|---|---|---|---|---|
| DHA | 160-240 | mg | 192 | 181 | 188 |
| EPA | 32-48 | mg | 39 | 37 | 35 |
| Vitamin B9 | 320-480 | μg | 369 | 352 | 324 |
| Vitamin B12 | 2.0-2.8 | μg | 2.5 | 2.6 | 2.2 |
| Vitamin B6 | 1.1-1.5 | mg | 1.2 | 1.3 | 1.4 |
| Vitamin B1 | 0.9-1.3 | mg | 1.1 | 1.2 | 1.1 |
| Vitamin B2 | 1.1-1.7 | mg | 1.2 | 1.5 | 1.4 |
| Vitamin B3 | 13-18 | mg | 15 | 16 | 14 |
| Vitamin B5 | 5-7 | mg | 6 | 6 | 5 |
| Vitamin B8 | 40-55 | μg | 47 | 49 | 44 |
| Vitamin C | 32-50 | mg | 34 | 32 | 41 |
| Vitamin D | 8-13 | μg | 10 | 10 | 10 |
| Y | 160-220 | μg | 180 | 171 | 201 |
| Fe | 22-31 | mg | 28 | 26 | 29 |
| Zn | 8-11 | mg | 10 | 10 | 10 |
| Se | 44-61 | μg | 49 | 55 | 48 |
| Choline | 74-90 | mg | 81 | 80 | 81 |

The filling composition of the capsules had the composition described in the following table after 0, 3, 6, 9, 12, 18 and 24 months storage in a long-term conditions chamber (25° C./60% RH Relative humidity (RH)):

| | Specification | | | | | | | | |
|---|---|---|---|---|---|---|---|---|---|
| Parameter | per capsule | | T0 | T3 | T6 | T9 | T12 | T18 | T24 |
| DHA | 160-240 | mg | 192 | 181 | 184 | 172 | 162 | 217.5 | 237.1 |
| EPA | 32-48 | mg | 39 | 38 | 35 | 35 | 37.8 | 37.2 | 48 |
| Vitamin B9 | 320-480 | μg | 369 | 356 | 401 | 426 | 352 | 398.6 | 392.5 |
| Vitamin B12 | 2.0-2.8 | μg | 2.5 | 2.6 | 2.2 | 2.3 | 2.2 | 2.2 | 2.10 |
| Vitamin B6 | 1.1-1.5 | mg | 1.2 | 1.4 | 1.4 | 1.2 | 1.3 | 1.26 | 1.22 |
| Vitamin B1 | 0.9-1.3 | mg | 1.1 | 1.2 | 1.1 | 1.2 | 1.1 | 1.06 | 0.98 |
| Vitamin B2 | 1.1-1.7 | mg | 1.2 | 1.5 | 1.5 | 1.6 | 1.3 | 1.21 | 1.17 |
| Vitamin B3 | 13-18 | mg | 15 | 16 | 14 | 14 | 15 | 14.02 | 13.84 |
| Vitamin B5 | 5-7 | mg | 6 | 7 | 5 | 7 | 6 | 5.61 | 5.53 |
| Vitamin B8 | 40-55 | μg | 47 | 50 | 49 | 48 | 46 | 47.3 | 45.9 |
| Vitamin C | 32-50 | mg | 34 | 32 | 40 | 38 | 42 | 40.21 | 39.89 |
| Vitamin D | 8-13 | μg | 10 | 11 | 10 | 11 | 12 | 10.8 | 10.3 |
| Y | 160-220 | μg | 180 | 182 | 177 | 203 | 200 | 194 | 180 |
| Fe | 22-31 | mg | 28 | 29 | 26 | 30 | 30 | 30.7 | 25 |
| Zn | 8-11 | mg | 10 | 10 | 9 | 11 | 10 | 10.8 | 11 |
| Se | 44-61 | μg | 49 | 58 | 51 | 53 | 49 | 54 | 45 |
| Choline | 74-90 | mg | 81 | 85 | 84 | 81 | 78 | 81.3 | 82 |

The filling composition of the capsules had the composition described in the following table after 0, 3, 6, 9, 12, 18 and 24 months of storage in a long-term conditions chamber (30° C./65% RH Relative humidity (RH)):

| | Specification | | | | | | | | |
|---|---|---|---|---|---|---|---|---|---|
| Parameter | per capsule | | T0 | T3 | T6 | T9 | T12 | T18 | T24 |
| DHA | 160-240 | mg | 192 | 180 | 194 | 183 | 165 | 222.7 | 220.5 |
| EPA | 32-48 | mg | 39 | 37 | 41 | 41 | 40.8 | 38.5 | 47.8 |
| Vitamin B9 | 320-480 | μg | 369 | 348 | 350 | 378 | 402 | 365.3 | 360.2 |
| Vitamin B12 | 2.0-2.8 | μg | 2.5 | 2.5 | 2.4 | 2.6 | 2.3 | 2.1 | 2.1 |
| Vitamin B6 | 1.1-1.5 | mg | 1.2 | 1.3 | 1.1 | 1.4 | 1.2 | 1.25 | 1.2 |
| Vitamin B1 | 0.9-1.3 | mg | 1.1 | 1.2 | 1.0 | 1.2 | 1.1 | 0.99 | 0.96 |
| Vitamin B2 | 1.1-1.7 | mg | 1.2 | 1.4 | 1.5 | 1.6 | 1.5 | 1.23 | 1.19 |
| Vitamin B3 | 13-18 | mg | 15 | 15 | 14 | 15 | 15.2 | 13.92 | 13.65 |
| Vitamin B5 | 5-7 | mg | 6 | 6 | 6 | 6 | 6 | 5.44 | 5.12 |
| Vitamin B8 | 40-55 | μg | 47 | 48 | 51 | 49 | 43 | 44.6 | 44.2 |
| Vitamin C | 32-50 | mg | 34 | 32 | 36 | 42 | 41 | 39.14 | 38.54 |
| Vitamin D | 8-13 | μg | 10 | 10 | 10 | 10 | 9.8 | 10.6 | 10.1 |
| Y | 160-220 | μg μg | 180 | 179 | 200 | 200 | 180 | 190 | 186 |
| Fe | 22-31 | mg | 28 | 28 | 29 | 30 | 25 | 28.2 | 26 |
| Zn | 8-11 | mg | 10 | 8 | 10 | 10 | 10 | 10 | 11 |
| Se | 44-61 | μg | 49 | 57 | 48 | 56 | 58 | 51 | 45 |
| Choline | 74-90 | mg | 81 | 80 | 81 | 79 | 82 | 80.6 | 78 |

Results showed that compounds of the capsules lasted at least six months stored in an accelerated conditions chamber at 40° C./75% RH and at least 24 months stored at (25° C./60% RH and 30°/65% RH) without significant changes.

In all cases the capsules maintained their color, smell, shape, disintegration time and weight per capsule over time. The peroxides index was below <5 mEq/kg O₂ in all cases.

In addition, microbiological test were satisfactorily passed in all cases. In particular, no Staphylococcus aureous, Escherichia coli, Salmonella, or Listeria monocytogenes were found.

All active ingredients were stable in all storage conditions. In particular, vitamins that are sensitive to humidity such as vitamin A, vitamin D3, vitamin B2, vitamin B5, vitamin B6, vitamin B12, were stable (i.e. their amount was maintained between their predefined range per capsule) even by being in a composition of a soft capsule with about 204 mg of GPC (that correspond to about 81 mg of choline)

### Example 6

### Stability Study of Vitamins in Soft Capsules Comprising Choline Glycerophosphate (GPC)

In the present Example, the stability over time and under different conditions of soft gelatin capsules comprising choline glycerophosphate as described in Example 10 was studied.

Once prepared, the capsules were packaged within an aluminium (20 microns) and PVC/PVDC (250/60 microns) blister packaging and the stability of the active compounds was tested by keeping the capsules in an accelerated conditions chamber (40° C./75% Relative humidity (RH)) and long-term conditions chambers (25° C./60% RH and 30°/65% RH) for several months.

The number of months “n” under accelerated condition storage is expressed as “Tn”; for Example T0 refers to the original composition at the beginning of the test, and T3 to 3 months of storage under accelerated conditions.

The filling composition of the capsules had the composition described in the following table after 0, 3 or 6 months storage in an accelerated conditions chamber (40° C./75% Relative humidity (RH)):

| | Specification | | | | |
|---|---|---|---|---|---|
| Parameter | per capsule | | T0 | T3 | T6 |
| DHA | 80-120 | mg | 96.4 | 104.6 | 91.7 |
| Vitamin B9 | 80-120 | μg | 93.1 | 110.9 | 111.2 |
| Vitamin B12 | 1.00-1.37 | μg | 1.10 | 1.10 | 1.18 |
| Vitamin B1 | 0.44-0.66 | mg | 0.62 | 0.65 | 0.45 |
| Vitamin B2 | 0.6-0.8 | mg | 0.72 | 0.73 | 0.66 |
| Vitamin B3 | 6-9 | mg | 8.2 | 6.6 | 6.0 |
| Vitamin B5 | 2.4-3.3 | mg | 3.22 | 3.08 | 2.66 |
| Vitamin B6 | 0.6-0.8 | mg | 0.77 | 0.77 | 0.79 |
| Vitamin B8 | 20-28 | μg | 24.1 | 24.9 | 24.8 |
| Vitamin A | 320-500 | μg | 413 | 446 | 442 |
| Vitamin C | 32-50 | mg | 48.68 | 42.92 | 41.37 |
| Vitamin D | 2.0-3.1 | μg | 2.7 | 2.9 | 3.0 |
| Vitamin E | 5-8 | mg | 7.44 | 5.04 | 7.02 |
| Taurine | 16-24 | mg | 19 | 21 | 21.3 |
| Cu | 400-550 | μg | 405,9 | 410 | 417 |
| Y | 80-110 | μg | 94.8 | 95 | 90 |
| Fe | 6-8 | mg | 7.51 | 6.35 | 6.07 |
| Zn | 4-6 | mg | 5.11 | 6.00 | 5.99 |
| Se | 8-11 | μg | 9.4 | 11 | 11 |
| Choline | 40-55 | mg | 48.2 | 46.2 | 45.6 |

The filling composition of the capsules had the composition described in the following table after 0, 3 or 6 months storage in a long-term conditions chamber (30° C./65% Relative humidity (RH)):

| | | | | |
|---|---|---|---|---|
| | Specification | | | |
| Parameter | per capsule | T0 | T3 | T6 |

| | | | | | |
|---|---|---|---|---|---|
| DHA | 80-120 | mg | 96.4 | 99.8 | 110.7 |
| Vitamin B9 | 80-120 | μg | 93.1 | 104.6 | 103.8 |
| Vitamin B12 | 1.00-1.37 | μg | 1.10 | 1.20 | 1.31 |
| Vitamin B1 | 0.44-0.66 | mg | 0.62 | 0.61 | 0.61 |
| Vitamin B2 | 0.6-0.8 | mg | 0.72 | 0.76 | 0.72 |
| Vitamin B3 | 6-9 | mg | 8.2 | 8.7 | 7.7 |
| Vitamin B5 | 2.4-3.3 | mg | 3.22 | 3.08 | 2.61 |
| Vitamin B6 | 0.6-0.8 | mg | 0.77 | 0.74 | 0.76 |
| Vitamin B8 | 20-28 | μg | 24.1 | 20.8 | 27.4 |
| Vitamin A | 320-500 | μg | 413 | 395 | 389 |
| Vitamin C | 32-50 | mg | 48.68 | 48.30 | 38.50 |
| Vitamin D | 2.0-3.1 | μg | 2.7 | 2.0 | 2.6 |
| Vitamin E | 5-8 | mg | 7.4 | 7.7 | 6.5 |
| Taurine | 16-24 | mg | 19 | 18 | 17.6 |
| Cu | 400-550 | μg | 405.9 | 400 | 417 |
| Y | 80-110 | μg | 94.8 | 90 | 90 |
| Fe | 6-8 | mg | 7.51 | 6.10 | 6.49 |
| Zn | 4-6 | mg | 5.11 | 5.65 | 5.90 |
| Se | 8-11 | μg | 9.4 | 11 | 9.5 |
| Choline | 40-55 | mg | 48.2 | 42.1 | 45.8 |

The filling composition of the capsules had the composition described in the following table after 0, 3 or 6 months storage in a long-term conditions chamber (25° C./60% Relative humidity (RH)):

| | | | | |
|---|---|---|---|---|
| | Specification | | | |
| Parameter | per capsule | T0 | T3 | T6 |

| | | | | | |
|---|---|---|---|---|---|
| DHA | 80-120 | mg | 96.4 | 99.8 | 105.2 |
| Vitamin B9 | 80-120 | μg | 93.1 | 105.4 | 119.0 |
| Vitamin B12 | 1.00-1.37 | μg | 1.10 | 1.20 | 1.29 |
| Vitamin B1 | 0.44-0.66 | mg | 0.62 | 0.62 | 0.60 |
| Vitamin B2 | 0.6-0.8 | mg | 0.72 | 0.72 | 0.73 |
| Vitamin B3 | 6-9 | mg | 8.2 | 8.8 | 7.5 |
| Vitamin B5 | 2.4-3.3 | mg | 3.22 | 3.04 | 2.62 |
| Vitamin B6 | 0.6-0.8 | mg | 0.77 | 0.71 | 0.77 |
| Vitamin B8 | 20-28 | μg | 24.1 | 22.4 | 27.5 |
| Vitamin A | 320-500 | μg | 413 | 405 | 461 |
| Vitamin C | 32-50 | mg | 48.68 | 48.11 | 32.10 |
| Vitamin D | 2.0-3.1 | μg | 2.7 | 2.4 | 3.0 |
| Vitamin E | 5-8 | mg | 7.44 | 6.05 | 7.46 |
| Taurine | 16-24 | mg | 19 | 18 | 21.2 |
| Cu | 400-550 | μg | 405.9 | 410 | 405 |
| Y | 80-110 | μg | 94.8 | 95 | 90 |
| Fe | 6-8 | mg | 7.51 | 7.00 | 6.10 |
| Zn | 4-6 | mg | 5.11 | 5.25 | 4.36 |
| Se | 8-11 | μg | 9.4 | 9.3 | 11 |
| Choline | 40-55 | mg | 48.2 | 45.1 | 46.1 |

Similar results as those in Example 5 were observed for vitamins sensitive to humidity in a composition of a soft capsule comprising about 123.46 mg after testing the stability of said capsule in all storage conditions mentioned above. Choline are provided as GPC in the samples.

In all cases the capsules maintained their color, smell, shape, disintegration time and weight per capsule over time in long-term conditions chambers. In addition, microbiological test were satisfactorily passed in all cases. In particular, no Staphylococcus aureous, Escherichia coli, Salmonella, or Listeria monocytogenes were found. All active ingredients were stable in all storage conditions. In particular, vitamins that are sensitive to humidity such as vitamin A, vitamin D3, vitamin B2, vitamin B5, vitamin B6, vitamin B12, were stable (i.e. their amounts was maintained between their predefined range per capsule) even by being in a composition of a soft capsule with about 123 mg of GPC (about 41 mg of choline).

1. A soft gelatin capsule comprising:
a) a soft gelatin capsule shell; and
b) a capsule filling composition comprising
between 50 and 1000 mg of choline glycerophosphate;
at least an essential unsaturated fatty acid; and
at least a humidity sensitive vitamin.

2. The soft gelatin capsule according to claim 1, wherein the at least a humidity sensitive vitamin is selected from the group consisting of vitamin A, vitamin C, vitamin D, vitamin B1, vitamin B2, vitamin B5, vitamin B6, vitamin B9, vitamin B12, vitamin K and combinations thereof.

3. The soft gelatin capsule according to claim 1, wherein the capsule filling composition further comprises an element selected from zinc, copper, iron and combinations thereof.

4. The soft gelatin capsule according to claim 1, wherein the capsule filling composition further comprises selenium.

5. The soft gelatin capsule according to claim 1, wherein the choline glycerophosphate is in at least an 8 wt % of the total weight of the soft gelatin capsule.

6. The soft gelatin capsule according to claim 1, wherein the at least an essential unsaturated fatty acid is at least an omega-3 fatty acid selected from a-linolenic acid (ALA), eicosapentaenoic acid (EPA), and docosahexaenoic acid (DHA), hexadecatrienoic acid (HTA), stearidonic acid (SDA), eicosatrienoic acid (ETE), eicosatetranoic acid (ETA), heneicosapentaenoic acid (HPA), docosapentaenic acid and mixtures thereof; preferably the at least an omega-3 fatty acid is docosahexaenoic acid (DHA).

7. The soft gelatin capsule according claim 1 wherein the at least an omega-3 fatty acid is a mixture of fatty acids comprising a weight ratio between 1:50 and 1:1.2 of EPA:DHA.

8. The soft gelatin capsule according to claim 1, wherein the capsule filling composition (b) comprises:
between 70 and 700 mg of choline glycerophosphate; and
between 100 and 1500 mg of fish oil comprising eicosapentaenoic acid (EPA) and docosahexaenoic acid (DHA); wherein the weight ratio of EPA:DHA is between 1:50 and 1:1.2.

9. The soft gelatin capsule according to claim 1, wherein the capsule filling composition further comprises an element selected from copper, iodine, calcium and combinations thereof.

10. The soft gelatin capsule according to claim 1, wherein the filling composition comprises:
between 70 and 700 mg of choline glycerophosphate;
between 100 and 1500 mg offish oil comprising eicosapentaenoic acid (EPA) and docosahexaenoic acid (DHA); wherein the weight ratio of EPA:DHA is between 1:50 and 1:1.2;
between 15 and 150 mg of ferrous fumarate or between 1 and 150 mg of ferrous bisglycinate chelate;
between 1 and 30 mg of zinc oxide;
optionally, between 0.5 and 5 mg of anhydrous copper sulfate or copper sulfate 1-hydrate;
between 10 and 200 pg of sodium selenite;
between 50 and 500 pg of potassium iodate;
optionally, 50 and 700 mg of calcium carbonate; and
between 0.05 and 3 mg of vitamin B1 ; or
between 0.3 and 5 mg of vitamin B2; or
between 1 and 15 mg of vitamin B5; or
between 0.1 and 5 mg of vitamin B6; or
between 50 and 1000 pg of vitamin B9 and/or 50 and 2000 pg of glucosamine salt of (6S)-5-methyltetrahydrofolate; or
between 0.5 and 10 pg of vitamin B12; or
between 1 and 50 pg of vitamin D3; and
optionally, between 100 and 1500 pg RE of vitamin A; and
optionally, between 10 and 300 mg of vitamin C.

11. The soft gelatin capsule according to claim 10, wherein the filling composition further comprises:
between 5 and 50 mg of vitamin B3;
between 10 and 100 pg of vitamin B8.

12. The soft gelatin capsule according to claim 11, wherein the filling composition further comprises:
between 1 and 30 mg of vitamin E; and
optionally, taurine.

13. The soft gelatin capsule according to claim 1, wherein the gelatin of the soft capsule shell is selected from gelatin A, gelatin B and mixtures thereof.

14. The soft gelatin capsule according to claim 1, wherein the soft gelatin capsule shell comprises a plasticizer selected from the group consisting of glycerin, sorbitol, propylene glycol, polyethylene glycol, dibutyl sebacate, diethyl phthalate, dimethyl phthalate, triacetin, tributyl citrate, triethyl citrate, and mixtures thereof.

15. (canceled)

16. (canceled)

17. A process for preparing the soft gelatin capsule as defined in claim 1, said process comprising:
a) preparing a capsule filling composition comprising: between 50 and 1000 mg of choline glycerophosphate, at least an essential unsaturated fatty acid; and at least a humidity sensitive vitamin;
b) forming a soft gelatin capsule shell;
c) filling the soft gelatin shell with the capsule filling composition of step a) to obtain a soft gelatin capsule, and
d) drying the soft gelatin capsule obtained in step c).

18. (canceled)

19. (canceled)

20. The soft gelatin capsule according to claim 1, wherein the at least an omega-3 fatty acid is docosahexaenoic acid (DHA).

21. A method
of treatment or prevention of a disease or pathology related to choline insufficiency, said method comprising administering to a patient in need of such a treatment or prevention a soft gelatin capsule as defined in claim 1; or
of diet supplementation, comprising administering to an individual a soft gelatin capsule as defined in claim 1.

22. Method according to claim 21, wherein the patient or individual is selected from a pregnant woman, a lactating woman, a fetus, an infant or children.
